(19) United States
(12) Patent Application Publication
(10) Pub. No.: US 2003/0139689 A1
(43) Pub. Date: Jul. 24, 2003

(54) High torque, low profile intravascular guidewire system

(76) Inventors: Leonid Shturman, Moscow (RU); Dmitrii Urjevich Prudnikov, Schelkovskoe Shosse (RU); Aleksi Filippovich Filippov, Borovskoe Shosse (RU)

Correspondence Address: Cummings & Lockwood, Four Stamford Plaza, 107 Elm Street, Stamford, CT, 06902, US

(21) Appl. No.: 10/223,527

(22) Filed: Dec. 23, 2002

Related U.S. Application Data
(60) Provisional application No. 60/338,354, filed on Nov. 19, 2001.
(60) Provisional application No. 60/334,297, filed on Nov. 30, 2001.

(51) Int. Cl. A61B 5/00

(52) U.S. Cl. 600/585

(57) ABSTRACT

A guidewire system is disclosed for performing a rotational atherectomy procedure, wherein the system includes a core wire having an elongated wire body defining a flexible distal portion and a more rigid medial portion, and a torquing sheath for positioning the core wire, the torquing sheath having a normally curved, relatively flexible distal portion and an interior lumen dimensioned and configured to accommodate the core wire, wherein the medial portion of the core wire is sufficiently rigid to straighten the normally curved distal portion of the torquing sheath when it is extended therethrough.

### CROSS REFERENCE TO RELATED APPLICATIONS

The subject application claims the benefit of priority of U.S. Provisional Patent Application Serial No. 60/338,354 filed Nov. 19, 2001 and U.S. Provisional Patent Application Serial No. 60/334,297 filed Nov. 30, 2001, the disclosures of which are herein incorporated by reference in their entireties.

### BACKGROUND OF THE INVENTION

1. Field of the Invention

The subject invention is related to intravascular surgical apparatus, and more particularly, to a high torque, low profile guidewire system for use in the performance of intravascular surgical procedures, and to methods of utilizing and manufacturing the same.

2. Background of the Related Art

Stenosis, which is a narrowing or obstruction of the interior lumen of a blood vessel, generally presents in patients suffering from atherosclerosis. This condition is characterized by an accumulation of fibrous, fatty or calcified tissue (atheromas) in the arteries. If left untreated, the stenosis can cause angina, hypertension, myocardial infarction, or strokes. Atheromas, also referred to as stenotic lesions, can be found at various sites in the arterial system, including the aorta, the coronary and carotid arteries, and peripheral arteries.

A variety of techniques and instruments have been developed for use in the ablation or removal of stenotic material. For example, U.S. Pat. No. 4,990,134 to Auth discloses a rotating burr covered with an abrasive cutting material and carried at the distal end of a flexible drive shaft. In use, when rotated at high speeds, the burr is used to remove stenotic material from an artery.

Rotational atherectomy devices, such as that which is disclosed in Auth, are used in conjunction with an elongated guidewire that directs the rotating drive shaft and burr through the arterial system. The guidewire plays a critical role is establishing the cutting vector of the device, as the burr will follow the course of the guidewire within an artery.

It is rare that a blood vessel is straight. More often, vessels are angled or tortuous, and guidewires have a tendency to project away from the central axis of the blood vessel. The divergence from the central axis of the vessel is referred to as guidewire bias. Guidewire bias can be problematic if it causes the burr to orient out of the plane of the vessel. This could result in radial or tangential ablation, or, even worse, perforation of the blood vessel.

Guidewires used with rotational atherectomy devices are commonly made from stainless steel monofilament, and are relatively stiff so as to prevent the guidewire from prolapsing or coiling during burr actuation, which could cause the guidewire to fracture. Guidewire bias often results from the relative stiffness of the guidewire. Some guidewires have a tapered distal end portion to make them more flexible. Flexibility is advantageous in negotiating tortuous blood vessels and reduces the risk of blood vessel perforation. However, the flexible distal end of a guidewire can have a tendency to whip during burr activation, making it difficult to control the position of the burr.

It would be beneficial to provide an improved guidewire system having sufficient rigidity to successfully navigate tortuous blood vessels and prevent prolapse, while, at the same time being sufficiently flexible so as not to cause damage to the vessel walls or reduce the ability to control burr orientation.

### SUMMARY OF THE INVENTION

The subject invention is directed to an intravascular guidewire system that includes a core wire having an elongated wire body defining a relatively flexible distal portion and a relatively rigid medial portion, and a torquing sheath for positioning the core wire. The torquing sheath has a normally curved, relatively flexible distal portion and an interior lumen dimensioned and configured to accommodate the core wire. The medial portion of the core wire is sufficiently rigid to straighten the normally curved distal portion of the torquing sheath when the medial portion of the core wire is extending therethrough. In contrast, when the relatively flexible distal portion of the core wire extends through the normally curved distal portion of the torquing sheath, the distal portion of the torquing sheath maintains its normally curved configuration.

The wire body is formed from a monofilament structure, and preferably from a metal alloy having shape memory characteristics. The distal portion of the wire body is heat-treated in such a manner so as to relieve the shape memory characteristics associated therewith. Furthermore, the distal portion of the wire body is conically tapered in a center-less grinding process, and is surrounded by a heat shrinkable polymeric sheath. The polymeric sheath extends beyond a distal end of the wire body to define a flexible tubular extension sleeve, wherein a clearance gap exist between the polymeric sheath and the distal-most section of the wire body.

A plurality of spaced apart radiopaque markers are disposed within the tubular extension sleeve. Preferably, the radiopaque markers include at least three generally cylindrical markers formed from a platinum and iridium alloy. Alternatively, a coiled radiopaque marker wire is disposed within the tubular extension sleeve. In addition, a plug is provided at the distal end of the tubular extension sleeve to seal the sleeve. The torquing sheath of the guidewire system of the subject invention is preferably constructed from plural helically wound layers of coiled wire filament. More particularly, the torquing sheath is constructed from an inner helically wound coil layer wound in a first direction and an outer helically wound coil layer wound in a second direction.

A tubular sleeve formed from a polymeric material is heat shrunk about the outer periphery of a proximal portion of the torquing sheath to form a sealing surface for interacting with a hemostasis valve. The tubular sleeve preferably extends along about 15% to 40% of the length of the torquing sheath. In an embodiment of the subject invention, a spherical member is secured to a distal end of the outer coil layer of the torquing sheath to render the distal end of the torquing sheath atraumatic to blood vessels.

It is envisioned that the normally curved distal portion of the torquing sheath has a reduced diameter to provide the curved distal portion with added flexibility and reduced rigidity relative to the remainder of the torquing sheath. Preferably, the reduction in diameter is accomplished by a chemical etching process. Furthermore, the distal portion of the torquing sheath is electrically treated so as to set the curvature therefor. Alternatively, the distal portion of the torquing sheath is defined by a flexible nosepiece having a radius of curvature. Preferably, the flexible nosepiece is formed from a polymeric material and includes a proximal portion for receiving the distal end of the torquing sheath.

The intravascular guidewire system of the subject invention further includes a rotatable ablation device including a drive shaft having an interior lumen and carrying an abrasive crown for ablating stenotic material, wherein the interior lumen of the drive shaft being dimensioned and configured to receive the core wire. In one embodiment of the subject invention, a monolithic tubular liner formed from a lubricious material is disposed within the interior lumen of the drive shaft to reduce friction between the core wire and the drive shaft. In another embodiment of the subject invention, a three layer tubular liner is disposed within the interior lumen of the drive shaft, wherein the liner has an outer layer formed from a material that bonds well to the interior surface of the drive shaft and an inner layer formed from a lubricious material that reduces friction between the guidewire and the drive shaft. In addition, the drive shaft has the eccentric section carrying the abrasive crown and a conically tapered section proximal to an eccentric shaft section.

The subject invention is also directed to a method of forming an intravascular guidewire which includes the steps of providing an elongated wire body formed from a material having shape memory characteristics, and treating a distal portion of the wire body in a manner so as to relieve the shape memory characteristics associated therewith. Preferably, the step of treating a distal portion of the wire body includes heating the distal portion of the wire body in an enclosure containing an inert gas for about thirty minutes at approximately 300° F.

The subject invention is also directed to a method of forming an intravascular torquing sheath that includes the steps of providing an elongated body formed from plural helically wound coil wire layers, positioning a distal portion of the elongated body over a cylindrical forming mandrel having a desired radius of curvature, and delivering an electrical current through the distal portion of the elongated body such that the distal portion assumes the curvature of the forming mandrel. The method further includes the step of reducing the outer diameter of the distal portion of the torquing sheath to enhance the flexibility thereof. This involves the steps of masking the distal portion of the torquing sheath and applying a chemical etching agent thereto.

These and other aspects of the intravascular guidewire system and the methods of manufacturing and using the same which are disclosed herein will become more readily apparent to those having ordinary skill in the art from the following description of the drawings taken in conjunction with the detailed description of the preferred embodiments of the invention.

### BRIEF DESCRIPTION OF THE DRAWINGS

So that those having ordinary skill in the art to which the subject invention pertains will more readily understand how to make and use the high torque, low profile intravascular guidewire system of the subject invention, preferred embodiments thereof will be described in detail hereinbelow with reference to the drawings, wherein:

FIG. 1 is a side elevation view, in cross-section, of a high torque, low profile intravascular guidewire system constructed in accordance with a preferred embodiment of the subject invention which includes a torquing sheath and a core wire that are used in conjunction with one another to navigate through the venous system of a patient in a highly controlled manner;

FIG. 2 is a side elevational view, in cross-section, of the distal portion of the guidewire system of FIG. 1, with the flexible distal portion of the core wire disposed within the normally curved distal end section of the torquing sheath;

FIG. 3 is a side elevational view, in cross-section, of the distal portion of the guidewire system of FIG. 1, with the relatively rigid medial section of the core wire disposed within the normally curved distal end section of the torquing sheath so that the distal end section of the torquing sheath is deflected into a relatively straight condition;

FIG. 4 is a side elevational view, in partial cross-section, of a torquing sheath constructed in accordance with a preferred embodiment of the subject invention wherein the diameter of the outer coil layer forming the curved distal section of the sheath is reduced to enhance flexibility;

FIG. 5 is a side elevational view, in partial cross-section, of another torquing sheath constructed in accordance with a preferred embodiment of the subject invention wherein the diameter of the outer coil layer forming the curved distal section and a portion of the sheath proximal to the curved distal section is reduced the diameter reduced to enhance flexibility;

FIG. 5A is a side elevational view, in partial cross-section, of a distal portion of another torquing sheath constructed in accordance with a preferred embodiment of the subject invention wherein a spherical member is associated with a distal end thereof to render the distal end of the torquing sheath atraumatic to blood vessels;

FIG. 6 is a side elevational view, in partial cross-section, of yet another torquing sheath constructed in accordance with a preferred embodiment of the subject invention wherein the of the sheath is defined by a flexible nose piece mounted to the distal end of the sheath;

FIG. 7 is a side elevation al view in cross-section of a core wire constructed in accordance with a preferred embodiment the subject invention wherein a distal portion of the core wire is conically tapered and includes an untapered distal end section, and a flexible sleeve is supported on the distal portion of the core wire by heat shrinking;

FIG. 7A is an enlarged localized view of the distal portion of the core wire illustrating a plurality of radiopaque markers supported within the flexible sleeve in axially spaced apart relationship, and the untapered distal end section of the core wire extends through the proximal-most marker;

FIG. 8 is a side elevation al view in cross-section of another core wire constructed in accordance with a preferred embodiment the subject invention wherein a distal portion of the core wire is conically tapered and has a flexible sleeve is supported on the distal portion of the core wire by heat shrinking;

FIG. 8A is an enlarged localized view of the distal portion of the core wire illustrating a plurality of radiopaque markers supported within the tubular sleeve in axially spaced apart relationship, and the conically tapered distal end section of the core wire extends through the proximal-most marker;

FIG. 9 is a side elevational view in cross-section of yet another core wire constructed in accordance with a preferred embodiment the subject invention wherein a distal portion of the core wire is conically tapered and includes an untapered distal end section, and a flexible sleeve is supported on the distal portion of the core wire by heat shrinking;

FIG. 9A is an enlarged localized view of the distal portion of the core wire illustrating a plurality of radiopaque markers supported within the flexible sleeve in axially spaced apart relationship, wherein the proximal-most marker is positioned on the conically tapered distal portion of the core wire and the untapped distal end section of the core wire extends through another marker;

FIG. 10 is an illustration of the aortic arch and associated blood vessels with a flexible guiding catheter extending therethrough and positioned such that the distal end of the catheter is disposed at the osteum of the coronary artery;

FIG. 11 illustrates the advancement of the guidewire system of the subject invention through the flexible catheter shown in FIG. 10;

FIG. 12 illustrates the advancement of the guidewire system of the subject invention into the coronary artery from the distal end of the guiding catheter disposed at the esteem of the coronary artery;

FIGS. 13 through 18 illustrate the navigation of the intravascular guidewire system of the subject invention within a blood vessel containing an eccentric stenotic lesion, wherein:

FIG. 13 illustrates the flexible distal end portion of the core wire projecting from the normally curved distal section of the torquing sheath disposed proximal to the lesion;

FIG. 14 illustrates the flexible distal end portion of the core wire partially advanced from the distal end of the torquing sheath;

FIG. 15 illustrates the flexible distal end portion of the core wire advanced from the distal end of the torquing sheath and positioned against the interior wall of the blood vessel;

FIG. 16 illustrates the torquing sheath and core wire rotated 180 degrees relative to the position illustrated in FIG. 15, to enable a more effective approach angle for traversing the lesion;

FIG. 17 illustrates the advancement of the core wire through the lesion; and

FIG. 18 illustrates the withdrawal of the torquing sheath from the site of the lesion, with the core wire remaining in an operational position in the blood vessel;

FIGS. 19 through 24 illustrate the navigation of the intravascular guidewire system of the subject invention within the venous system of a patient in such a manner so as to gain forward entry into a branch vessel containing an eccentric stenotic lesion, wherein:

FIG. 19 illustrates an initial orientation of the normally curved distal section of the torquing sheath as it approaches the opening to the branch vessel;

FIG. 20 illustrates the normally curved distal section of the torquing sheath rotated 180 degrees relative to the orientation shown in FIG. 19 to enable a more effective approach angle into the opening of the branch vessel;

FIG. 21 illustrates the advancement of the normally curved distal section of the torquing sheath into the opening of the branch vessel;

FIG. 22 illustrates the advancement of the core wire from the distal end of the torquing sheath into the branch vessel to a location proximal to the lesion;

FIG. 23 illustrates the core wire traversing the eccentric lesion within the branch vessel such that the distal end of the core wire is positioned beyond the lesion; and

FIG. 24 illustrates the withdrawal of the torquing sheath from the site of the lesion, with the core wire remaining in an operational position in the branch vessel;

FIGS. 25 through 32 illustrate the navigation of the intravascular guidewire system of the subject invention within the venous system of a patient in such a manner so as to gain a rearward entry route into a branch vessel containing an eccentric stenotic lesion, wherein:

FIG. 25 illustrates an initial orientation of the normally curved distal section of the torquing sheath as it approaches the opening to the branch vessel;

FIG. 26 illustrates the advancement of the core wire from the torquing sheath a sufficient distance so as to cause the relatively rigid medial portion of the core wire to partially straighten the normally curved distal section of the torquing sheath;

FIG. 27 illustrates the advancement of the partially straightened distal section of the sheath beyond the opening to the branch vessel;

FIG. 28 illustrates the withdrawal of the core wire within the lumen of the torquing sheath to a location which enables the distal section of the torquing sheath to return to its normally curved configuration distal to the opening of the branch vessel;

FIG. 29 illustrates the curved distal section of the torquing sheath as it approaches the opening of the branch vessel by moving proximally relative to the position shown in FIG. 28;

FIG. 30 illustrates the curved distal section of the torquing sheath positioned at the opening to the branch vessel;

FIG. 31 illustrates the advancement of the core wire from the distal end of the torquing sheath into the branch vessel and through the lesion contained therein; and

FIG. 32 illustrates the withdrawal of the torquing sheath from the site of the lesion, with the core wire remaining in an operational position in the branch vessel;

FIG. 33 is a perspective view of a prior art rotational atherectomy system that includes a burr having an abrasive surface which is mounted at the distal end of a drive shaft extended over an elongated guidewire having a flexible safety spring at the distal end thereof,

FIG. 34 is an enlarged side elevational view, in cross-section of the burr and distal portion of the guide wire of the system illustrated in FIG. 33, with the burr is a position proximal to the safety spring;

FIG. 35 is an enlarged side elevational view, in cross-section of the burr and distal portion of the guide wire of the system illustrated in FIG. 33, with the burr moved distally relative to the position shown in FIG. 34, so that it is in close proximity to the safety spring;

FIG. 36 is a perspective view of a rotational atherectomy system that includes a an eccentric drive shaft with an abrasive crown positioned over a prior art guidewire having a distal safety spring associated therewith;

FIG. 37 is an enlarged side elevational view, in cross-section, of the eccentric drive shaft of the system illustrated in FIG. 36, with the flexible distal end of the drive shaft positioned proximal to the safety spring;

FIG. 38 is an enlarged side elevational view, in cross-section, of the eccentric drive shaft of the system illustrated in FIG. 36, with the flexible distal end of the drive shaft moved distally relative to the position shown in FIG. 37, so that it is in close proximity to the safety spring;

FIG. 39 is an enlarged side elevational view, in cross-section, of the eccentric drive shaft of the system illustrated in FIG. 36, with the flexible distal end of the drive shaft positioned proximal to the flexible distal end portion of the core wire of the subject invention;

FIG. 40 is an enlarged side elevational view, in cross-section, of the eccentric drive shaft of the system illustrated in FIG. 36, with the flexible distal end of the drive shaft moved distally relative to the position shown in FIG. 39, so that it extends over the flexible distal end portion of the core wire of the subject invention;

FIG. 41 is an enlarged side elevational view, in cross-section, of the eccentric drive shaft of the system illustrated in FIG. 36, with the flexible distal end of the drive shaft moved distally relative to the position shown in FIG. 40, so that it extends beyond the flexible distal end portion of the core wire of the subject invention;

FIG. 42 is a side elevational view of a distal portion of the two layer torquing sheath of the subject invention prior to the application of any manufacturing processes thereon;

FIG. 43 illustrates the method of forming the curved distal section of the torquing sheath by way of an electrochemical treatment;

FIG. 44 illustrates the method of forming the curved distal section of the torquing sheath way of heat treatment;

FIG. 45 illustrates the curved distal section of the torquing sheath, with the inner and outer coil layers joined to one another, and the wherein the diameter of the outer coil layer is constant as compared to the reduced diameter shown in FIG. 4;

FIG. 46 illustrated the masking procedures performed prior to the performance of a chemical etching process which reduced the diameter of the outer coil layer of the torquing sheath;

FIG. 47 illustrates the method of heat treating the tapered distal portion of the core wire to alter the shape memory characteristics thereof;

FIG. 48 illustrates the core wire of the subject invention wherein the distal end of the flexible tube has a plug therein positioned adjacent to the distal-most radiopaque marker; and

FIG. 48A is an enlarged localized view of the distal portion of the core wire illustrated in FIG. 48;

FIG. 49 is a side elevational view of still another core wire constructed in accordance with a preferred embodiment of the subject invention wherein a solid marker is disposed within the flexible tubing positioned at the distal end of the core wire;

FIG. 49A is an enlarged localized view of the distal portion of the core wire illustrated in FIG. 49;

FIG. 50 is a side elevational view of another core wire constructed in accordance with a preferred embodiment of the subject invention wherein a monofilar coiled marker wire is supported within the flexible tubing positioned at the distal end of the core wire;

FIG. 50A is an enlarged localized view of the distal portion of the core wire illustrated in FIG. 50;

FIG. 51 is a perspective view of a prior art rotational atherectomy system that includes an eccentric drive shaft with an abrasive crown positioned over a prior art guidewire that includes a core wire having a tapered distal end portion and a polymeric outer sheath;

FIG. 51A is an enlarged side elevational view in cross-section of the eccentric shaft section of the drive shaft of FIG. 51 wherein the distal section of the drive shaft is retracted from the distal section of the guidewire;

FIG. 51B is an enlarged side elevational view in cross-section of the eccentric shaft section of the drive shaft of FIG. 51 wherein the distal section of the drive shaft surrounds the distal section of the guidewire;

FIG. 52 is an enlarged side elevational view in cross-section of an eccentric drive shaft constructed in accordance with a preferred embodiment of the subject invention which includes a monolithic tubular liner formed from a lubricious material and disposed within the interior lumen of the drive shaft to reduce friction between the guidewire and the drive shaft;

FIG. 53 is an enlarged side elevational view in cross-section of an eccentric drive shaft constructed in accordance with a preferred embodiment of the subject invention which includes a three layer tubular liner having an outer layer formed from a material that bonds well to the interior surface of the drive shaft and an inner layer formed from a lubricious material that reduces friction between the guidewire and the drive shaft;

FIG. 54 is an enlarged side elevational view in cross-section of an eccentric drive shaft constructed in accordance with a preferred embodiment of the subject invention wherein the drive shaft has a conically tapered section proximal to the eccentric shaft section and a monolithic tubular liner formed from a lubricious material is disposed within the interior lumen of the drive shaft to reduce friction between the guidewire and the drive shaft;

FIG. 55 is an enlarged side elevational view in cross-section of an eccentric drive shaft constructed in accordance with a preferred embodiment of the subject invention wherein the drive shaft has a conically tapered section proximal to the eccentric shaft section and a three layer tubular liner is disposed within the interior lumen of the drive shaft that includes an outer layer formed from a material that bonds well to the drive shaft and an inner layer formed from a lubricious material that reduces friction between the guidewire and the drive shaft;

FIG. 56 is a side elevational view in cross-section of a mandrel used to form the eccentric drive shaft of FIGS. 54 and 55; and

These and other features of the intravascular guidewire system of the subject invention and the methods of utilizing and manufacturing the same will become more readily apparent to those skilled in the art from the following detailed description of the preferred embodiments of the invention.

### DETAILED DESCRIPTION OF PREFFERED EMBODIMENTS

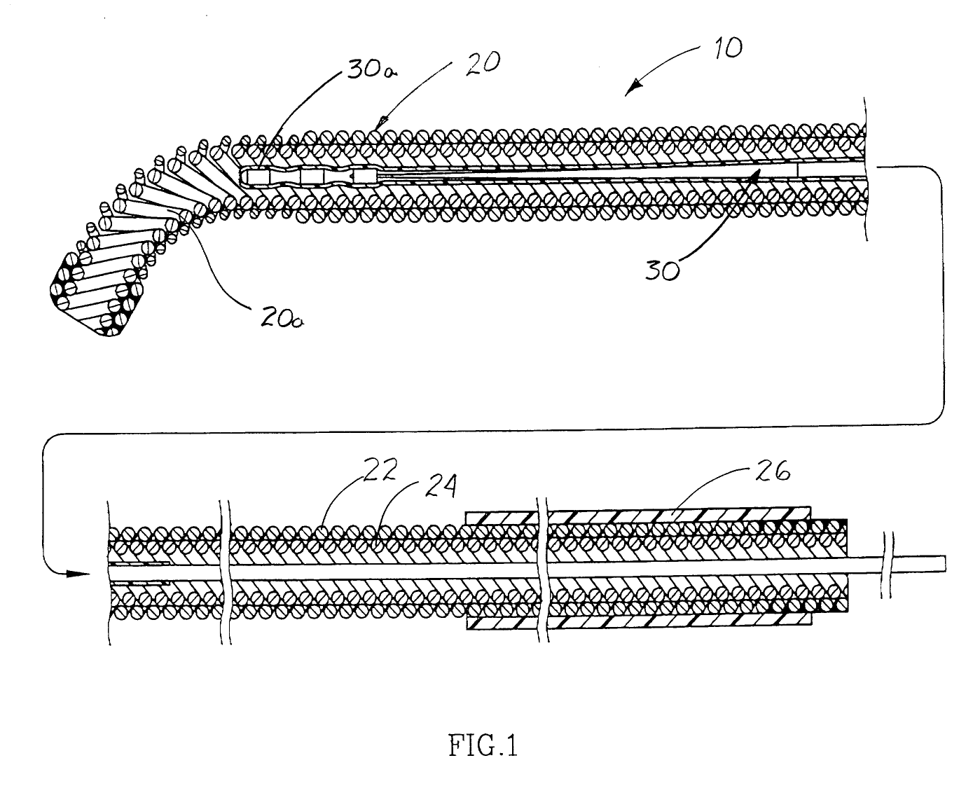

Referring now to the drawings wherein like reference numerals identify similar structural aspects of the guidewire system of the subject invention, there is illustrated in FIG. 1 an intravascular guidewire system constructed in accordance with a preferred embodiment of the subject invention and designated generally by reference numeral 10. Guidewire system 10 includes two primary components in the form of an elongated torquing sheath 20 having a curved distal section 20a and an elongated core wire 30 having a flexible distal portion 30a. The two components interact with one another in a unique manner so as to provide a guidewire system with a high degree of control and reliability.

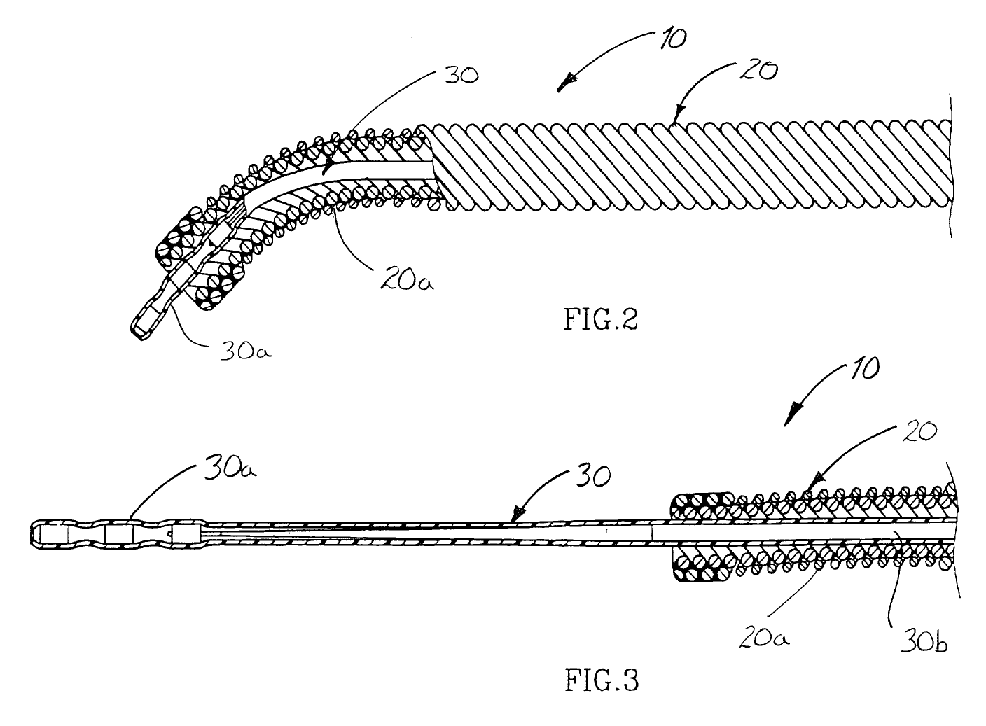

More particularly, as shown in FIG. 2, when the flexible distal portion 30a of the core wire 30 is positioned within the normally curved distal section 20a of sheath 20, the distal section of the sheath retains its normal radius of curvature. In contrast, as shown in FIG. 3, when the core wire 30 is advanced from the distal end of the sheath 20 such that the more rigid medial portion 30b of core wire 30 extends through the distal section 20a of the sheath 20, the rigid medial portion 30b of core wire 30 causes the normally curved distal section 20a to straighten. This enables the guidewire system of the subject invention to be navigated through circuitous blood vessels to access a broad range of stenotic lesions.

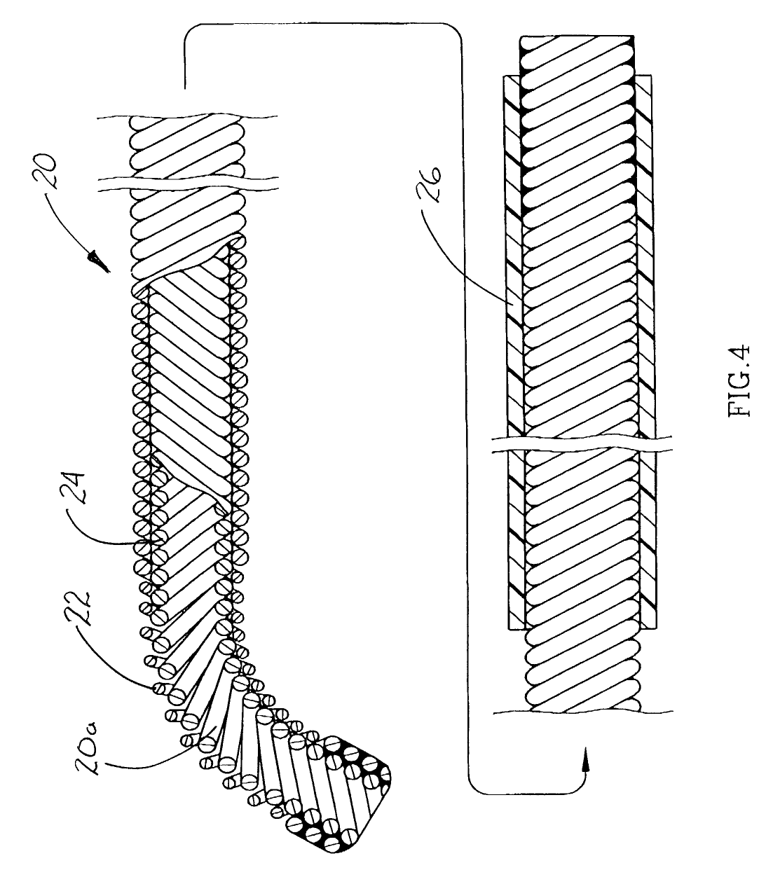

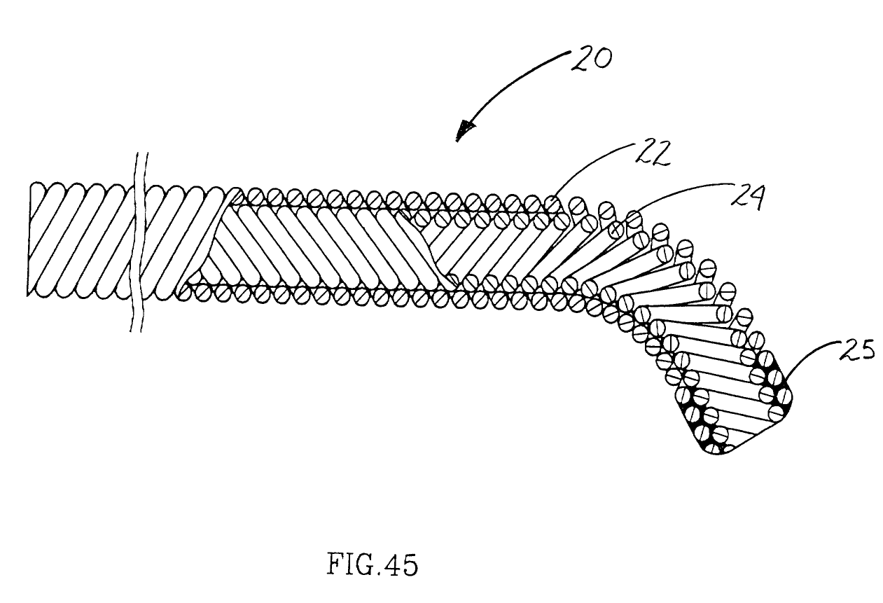

Referring now to FIG. 4, the elongated torquing sheath 20 of the subject invention is constructed from at least one helically wound coil, and preferably from a pair of helically wound coils that define an outer coil layer 22 and an inner coil layer 24. The two coil layers 22, 24 are formed from between one and five filars or wires of equal diameter. Preferably, the wire diameter is about 0.0035 inches. Those skilled in the art will readily appreciate that the diameter of the wire will have an effect on the stiffness and torqueability of the sheath. Suitable coils are available from Vadnais Technologies Corporation of St. Paul, Minn. Preferably, the coil layers 22, 24 are secured to one another at the opposed proximal and distal end regions of the sheath by soldering, welding or a similar joining technique known in the art, as discussed in more detail hereinbelow with reference to FIG. 45.

Preferably, the outer coil layer 22 is helically wound in a direction that is opposite that of the inner coil layer 24. More particularly, the helix of the outer coil layer 22 may be wound in a left hand direction, while the helix of the inner coil layer 24 may be wound in a right hand direction, or vise versa. As a result when the torquing sheath 20 is rotated in the direction of the wrap of the outer coil layer, the outer coil layer will tend to become more tightly wound, while the inner coil layer 24 will tend to unwind. This interaction between the coil layers provides enhanced torque in one rotational direction. In contrast, if the sheath was constructed from three helically wound layers, which is well within the scope of the subject disclosure, and the outer and inner layers were wound in the same direction, while the medial layer was wound in an opposite direction, the sheath would exhibit a high degree of torque in both rotational directions. Of course, it must be recognized that the use of three or more coil layers, while within the scope of this disclosure, increases the overall profile of the torquing sheath as well as cost, and thus two layers is most preferable.

As illustrated in FIG. 4, a tubular sleeve 26 preferably formed from polyester or a similar polymeric material is heat shrunk about the outer periphery of a proximal portion 20c of torquing sheath 20. Sleeve 26 provides a firm sealing surface for interacting with the sealing ring structure of a hemostasis valve (not shown). This prevents blood loss during a procedure. Hemostasis valves often include ports for injecting contrast fluid into the lumen of a guiding catheter to aide visualization. Preferably, sleeve 26 extends along about between 15% to 40% of the torquing sheath 20. A suitable heat shrinkable polyester tubing is available from Advanced Polymers Incorporated of Salem, N.H. This material has a heat shrinking temperature of 205° C. It is also envisioned that the outer layer of the torquing sheath could be formed from one or more wires that are already provided with a PTFE of hydrophilic coating.

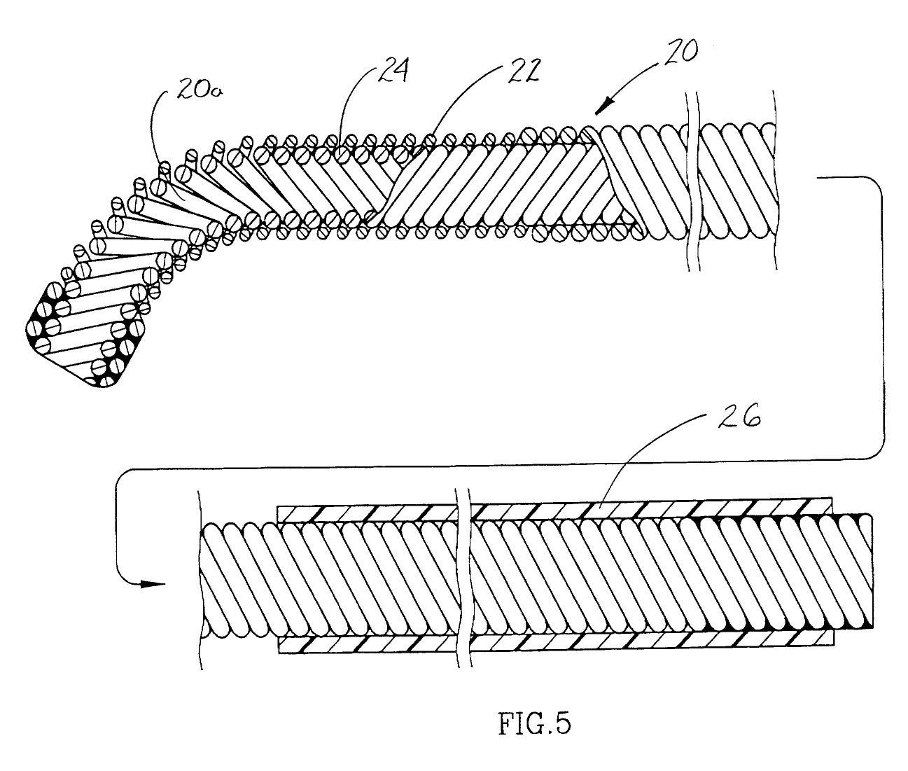

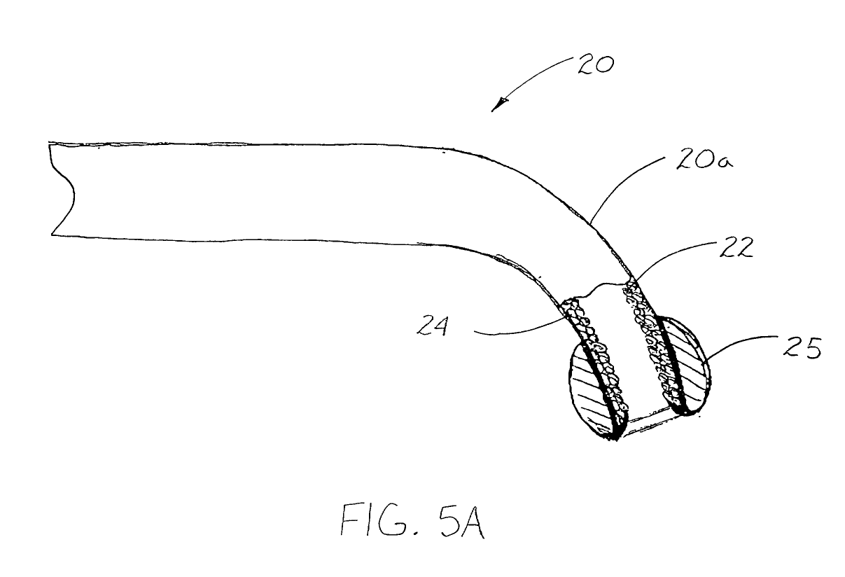

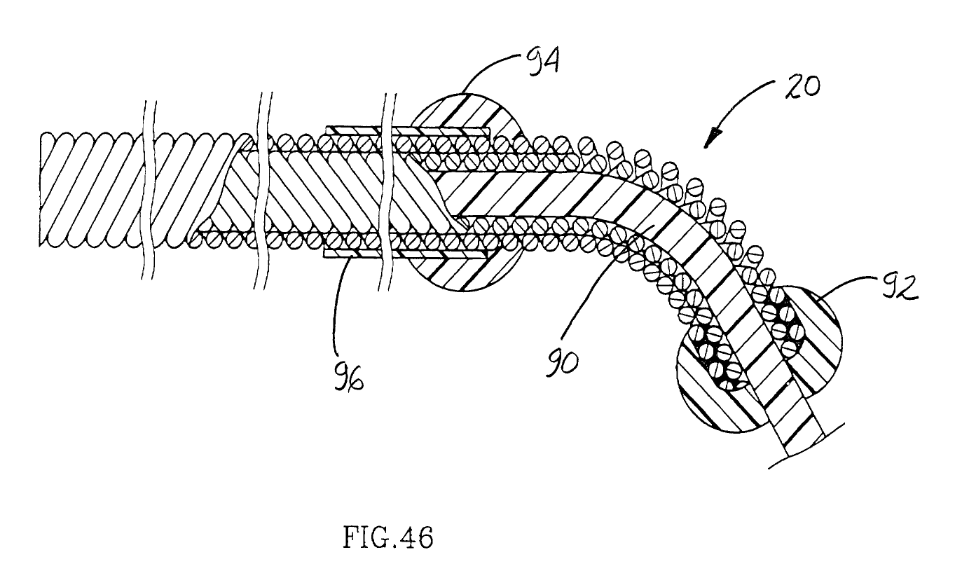

Referring to FIG. 4, as illustrated the curved distal section 20a of sheath 20 has a reduced diameter to provide the curved distal section 20a of sheath 20 with added flexibility and reduced rigidity. This is particularly useful in instances where the radius of curvature is relatively tight, and is generally unnecessary in instances where the radius of curvature is greater. As shown in FIG. 4, it is preferable that the outer coil layer 22 of the sheath is reduced in diameter while the inner coil layer 24 of the sheath retains its original diameter. FIG. 5 illustrates another instance wherein the curved distal section 20a of sheath 20 as well as a straight portion of the sheath proximal thereto has a reduced diameter. As explained in more detail hereinbelow with reference to FIG. 46, the reduction in the diameter of the outer coil layer 22 is accomplished by a chemical etching process.

As best seen in FIG. 5A, it is envisioned that a spherical member 25 is secured to a distal end of the outer coil layer 22 of the sheath by soldering or a similar fixation method to render the distal end of the torquing sheath atraumatic to blood vessels. The spherical member 25 is preferably formed at least in part from gold or similar precious metal that is approved for medical use, such as platinum or alloys thereof.

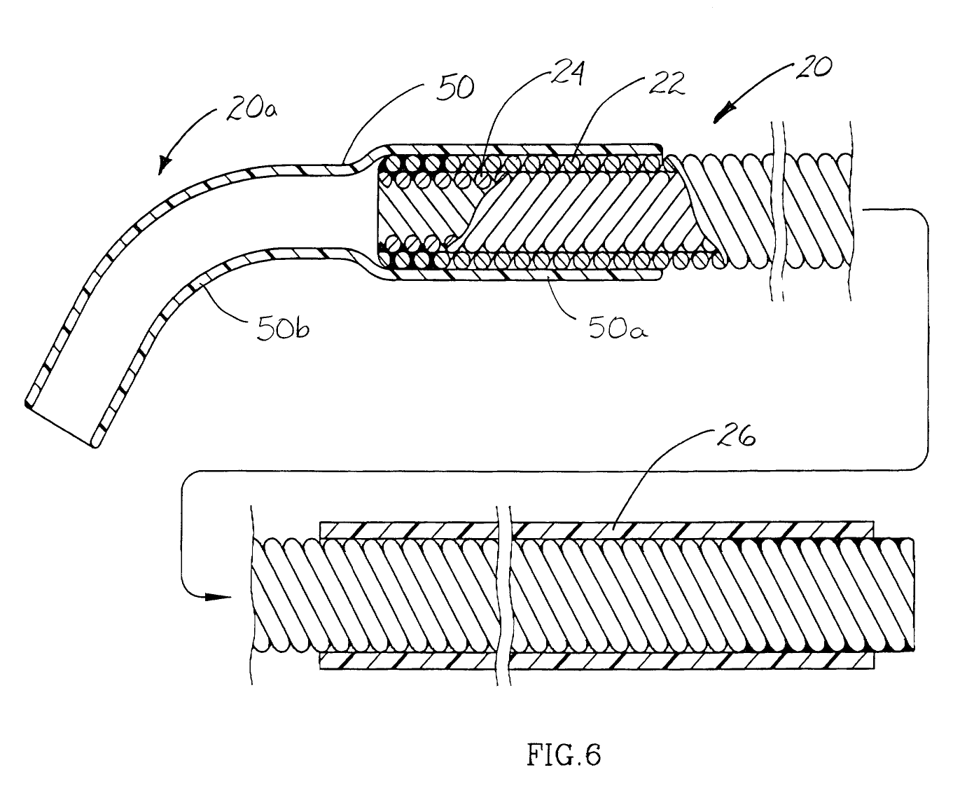

Referring to FIG. 6, in accordance with a preferred embodiment of the subject invention, the curved distal section 20a of torquing sheath 20 is defined by a flexible nosepiece 50. Nosepiece 50 is preferably formed from a polymeric material and includes a proximal sleeve portion 50a for receiving the distal end of the sheath 20, and a distal section 50b made up of a relatively straight distal segment, a curved medial segment and a relatively straight proximal segment.

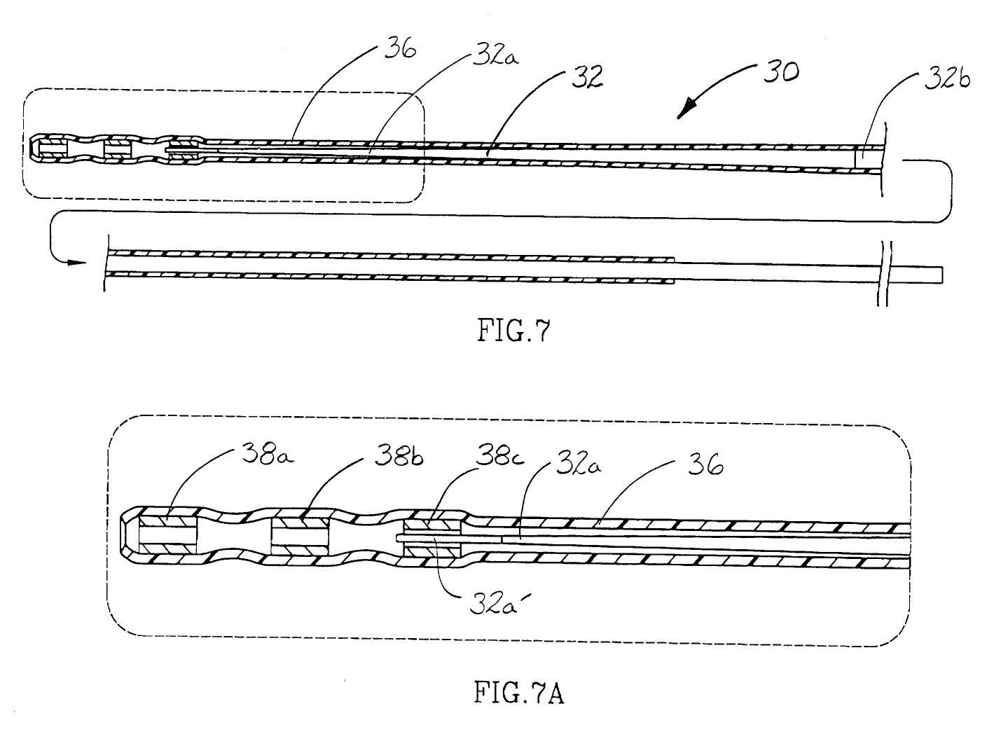

Referring now to FIG. 7, core wire 30 is defined by an elongated wire body 32 having a length of about 325 cm. A distal portion 32a of wire body 32 (about between 1.5 to 4.0 in., and preferably about between 2.5 and 3.0 in.) is conically tapered in a center-less grinding process so that the untapered portion 32b of wire body 32 has a diameter of about 0.009 in., while the distal portion 32a of the wire body 32 tapers to a diameter of about 0.005 in. A company that can perform the center-less grinding of wire body 32 is Wytech Industries Inc., of Rahway, N.J. As illustrated, there is a substantially smooth transition from the between the untapered and tapered portion of wire body 32. The tapering of the wire body 32 enhances the flexibility of the distal portion 30a of core wire 30, thereby reducing guidewire bias and a limiting fatigue related stress fractures.

The wire body 32 is preferably a monofilament structure formed from a metal alloy having shape memory characteristics, such as a nickel-titanium alloy (nitinol) or a similar super-elastic memory metal. This material is sufficiently rigid to facilitate navigation through the venous system of a patient and to prevent prolapsing (i.e., folding upon itself) or coiling during an intravascular procedure. In other words, the wire body 32 of core wire 30 is formed from a material providing a high degree of axial pushability.

To enhance the flexibility and thus reduce the bias that often results from a relative stiff guidewire, a distal portion of the wire body 32 is heat-treated in such a manner so as to relieve the shape memory characteristics associated therewith. As a result, the distal portion of the wire body is 32 more compliant and less brittle than the untreated portion of the wire body. The heat-treated portion is therefore more fatigue resistant than the untreated portion of the wire body 32. Furthermore, the bias that is often associated with a relatively stiff guidewire is significantly reduced, as is the tendency for the distal portion of the guidewire to “whip” during a rotational ablation procedure.

With continuing reference to FIG. 7, during an atherectomy procedure, a drive shaft is rotated about the axis of a guidewire at an extremely high rate of speed. This causes extreme heat and friction, and can result in fatigue fractures. It is commonplace therefore to introduce a saline cooling/lubricating solution between the relatively rotating components. An example of such a solution is one that is available from Boston Scientific and marketed under the tradename Rotaglide™ Lubricant.

The core wire 30 of the subject invention is adapted and configured in such a manner so that a special lubricating solution such as Rotaglide™ Lubricant is wholly unnecessary. In particular, a distal portion of wire body 32 is surrounded by a lubricious polymeric sleeve 36. The sleeve creates has a low coefficient of friction between the outer surface of the wire body 32 and the interior lumenal surface of a drive shaft. This will reduce stress on the wire body, and prevent fatigue fractures. The polymeric sleeve 36 is preferably formed from heat shrinkable polymer tubing, such as, for example, heat shrinkable polytetrafluoroethylene (PTFE) tubing. A suitable heat shrinkable PTFE tubing is available from Zeus of Raritan, N.J., which has a heat shrinking temperature of between 350° C. and 360° C. and an inner diameter of about 0.011 in. prior to heat shrinking and a maximum inner diameter of about 0.005 in. after heat shrinking. Those skilled in the art will readily appreciate that the material from which the PTFE tubing is formed must have a heat shrinking temperature range that does not interfere with the heat treatment temperature of shape memory metal from which the core wire 32 is formed.

As illustrated in FIG. 7A, polymer sleeve 36 extends about between 1 to 2 cm beyond the tapered distal end 32a of wire body 32 to define a flexible atraumatic tip for core wire 30. The inner diameter of the heat shrinkable polymer sleeve 36 (about 0.005 in.) is greater than the outer diameter of the distal-most section of the conically tapered portion 34a (0.002-0.003 in.) of the wire body 32. Therefore, a clearance gap exists between the two structures, as best seen in FIG. 7A. This gap acts to dampen vibrations in the wire body 32 during a rotational ablation procedure. It is envisioned that the flexible distal portion of the tubing extending beyond the wire body could be shaped in manner so as to have a preset curvature if so desired.

Referring to FIG. 7A, a train of three spaced apart, generally cylindrical, radiopaque markers 38a-38c are positioned within sleeve 36. The markers aide in the locational positioning of the distal end portion of core wire 30 within the vasculature of a patient when viewed by fluoroscopy. The number of markers can vary as well as their relative spacing. In this instance, the markers are substantially equidistant from one another, set at about 1.5 to 2.0 mm apart. Those skilled in the art will readily appreciate that the markers must be spaced relative to one another in such a manner so as to maintain visualization at any orientation with the venous system of a patient, and at the same time to provide the distal portion of sleeve 36 with sufficient flexibility. As illustrated, the outer diameter of each of the markers 38a-38c is greater than the minimal inner diameter of the interior lumen of sleeve 36. Thus, the heat shrinkable sleeve 36 secures the relative positions of the markers. The markers are preferably formed from a radiopaque material such as a platinum and iridium alloy. Suitable markers are available from Noble-Met, Ltd. of Salem, Va. These markers have a length of 0.5 mm, an outer diameter of about 0.0085 in. and an inner diameter of about 0.003 in.

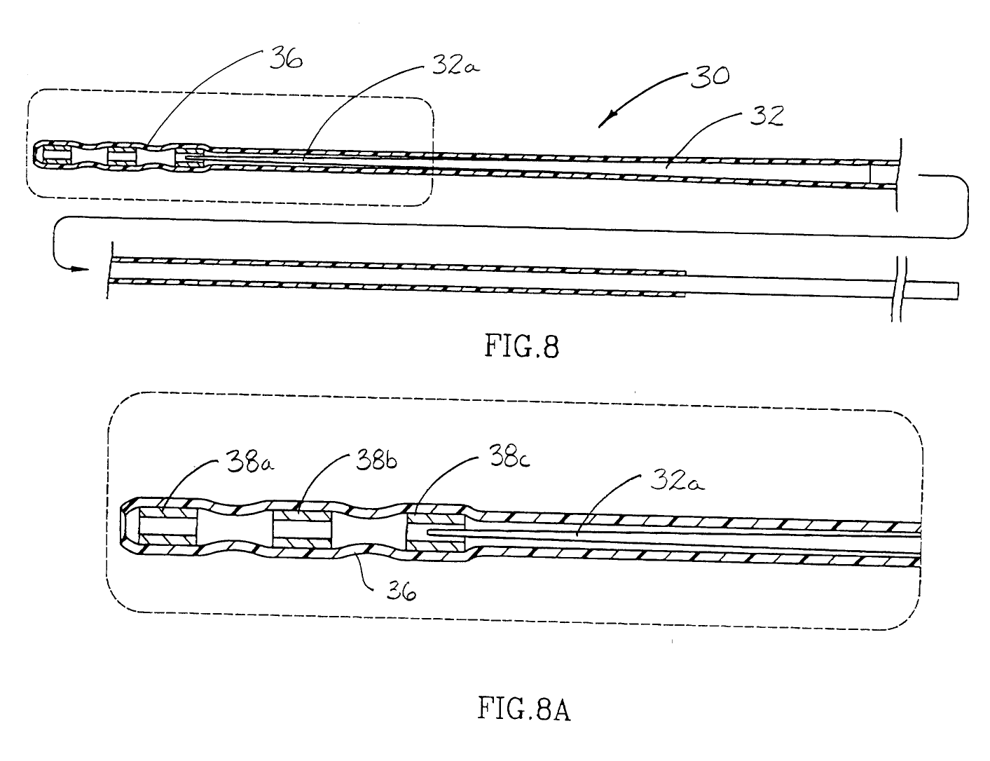

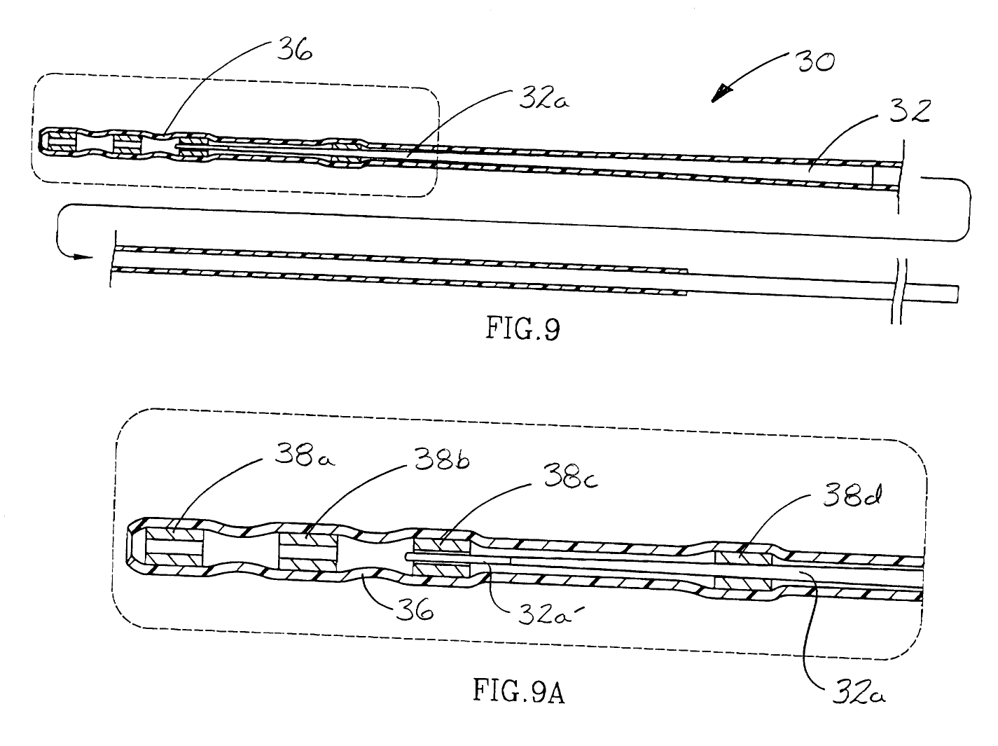

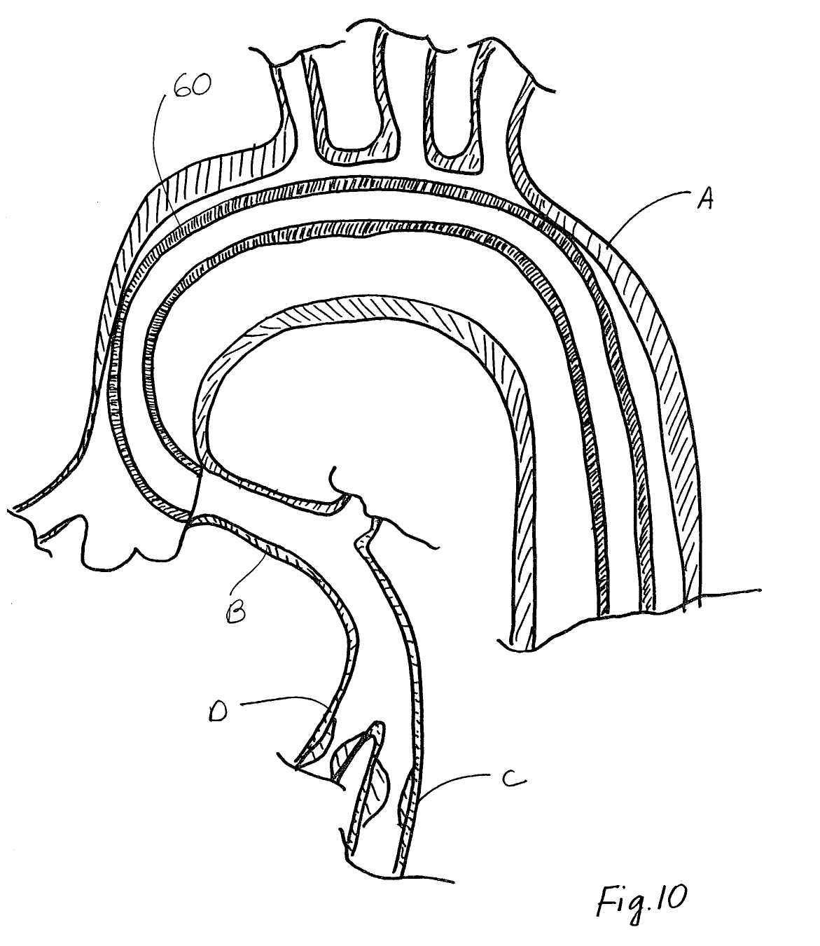

With continuing reference to FIG. 7A, as a consequence of the center-less grinding process used to form the conical taper of the core wire 32, a nose section 32a′ is formed a the distal end of the core wire. The nose is about 0.5 to 1.0 cm in length and can either be sheared off or left. If left, the nose would extend freely through the bore of the proximal-most marker 38c, as shown in FIG. 7A. Alternatively, if the nose is cut off, the distal tip of the conically tapered end would extend freely through the bore of the proximal-most marker 38c, as illustrated in FIGS. 8 and 8A. In an another embodiment of the core wire 30 of the subject invention, a fourth marker 38d is positioned on the conically tapered portion 32a of the core wire 32, as illustrated in FIGS. 9 and 9A. By leaving the distal-most end of the core wire 32 free from attachment with a marker, greater flexibility is achieved in the guidewire 30.

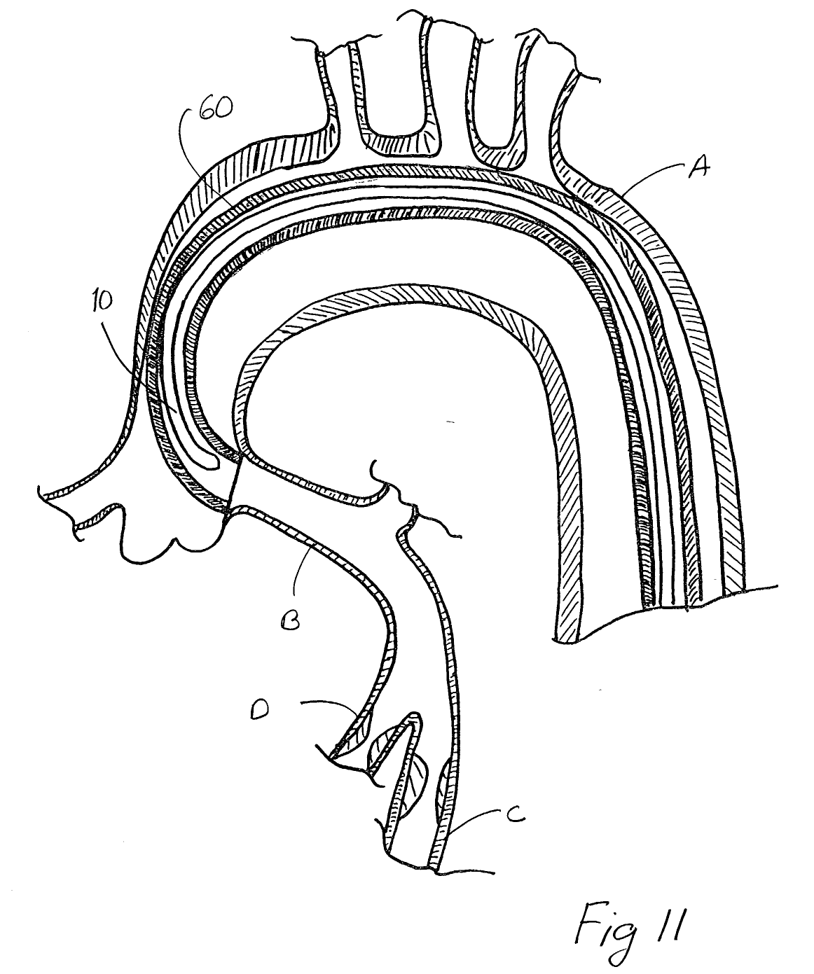

Referring now to FIG. 10, there is illustrated a section of the vasculature commonly referred to as the aortic arch A which is comprised mainly of the ascending and descending aorta. The left main branch B of the coronary artery extends from the ascending aorta and leads to the left anterior descending coronary artery C which bifurcates into the marginal branch of the coronary artery D. The left anterior descending coronary artery C and the marginal branch of the coronary artery D are common sites of stenotic lesions, and are usually difficult to approach with a conventional guidewire system for obvious anatomical reasons. However, the guidewire system 10 of the subject invention is particularly well adapted to gain access to lesions in these difficult to reach blood vessels. More particularly, as shown in FIG. 10, a guiding catheter 60 is extended through the aortic arch so that the curved distal end of the catheter is positioned at the osteum or opening of the coronary artery.

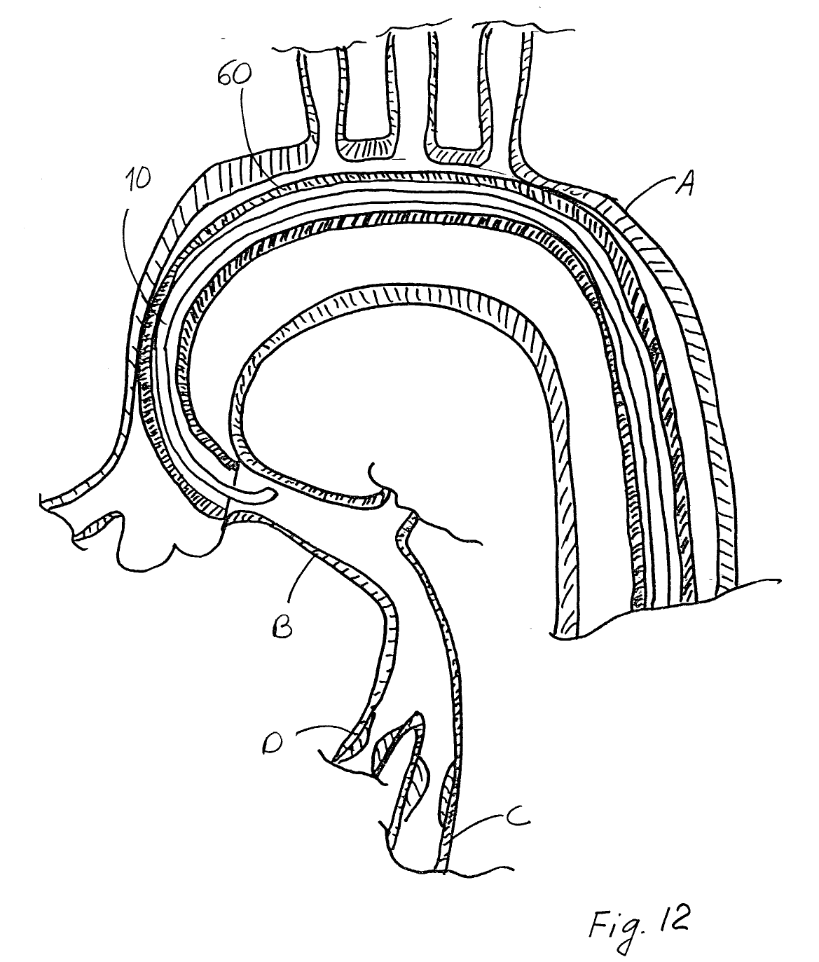

As is well known in the art, guiding catheters have been developed with curved distal end sections that are specifically configured in such a manner so as to approach either the left main branch of the coronary or the right coronary artery. Once the guiding catheter 60 is in place, the guidewire system 10 of the subject invention is advanced therethrough, as shown in FIG. 11. Then, as shown in FIG. 12, the guidewire system 10 is easily advanced into the coronary artery from the distal end of the guiding catheter. Once in the coronary artery, the distal end of the torquing sheath 20 may be easily advanced to the site of either of the two lesions by altering the curvature of the distal end of the torquing sheath using the core wire. Once located at the site of a lesion, the core wire 30 may be therethrough and the sheath may be withdrawn so that the remaining core wire can be used to guide an intravascular device to the site of the lesion.

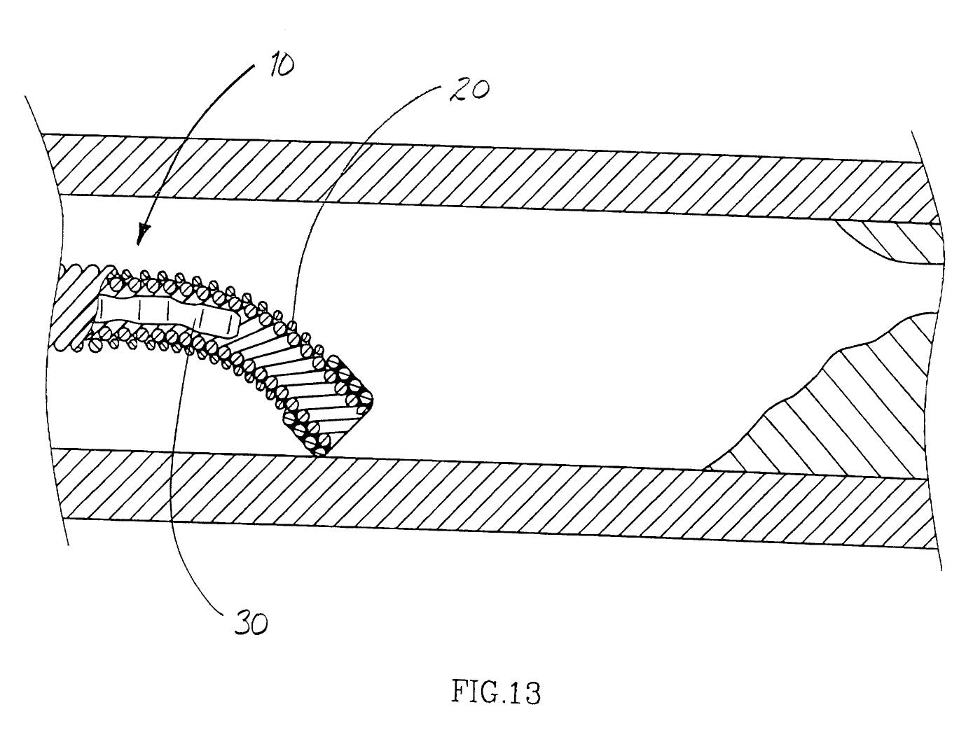

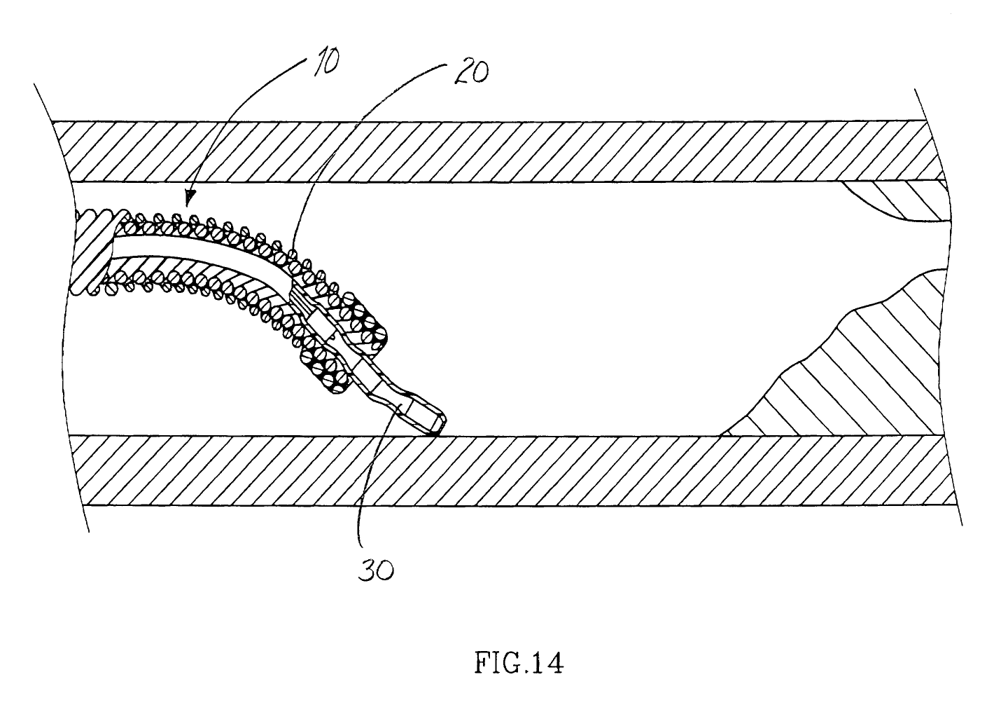

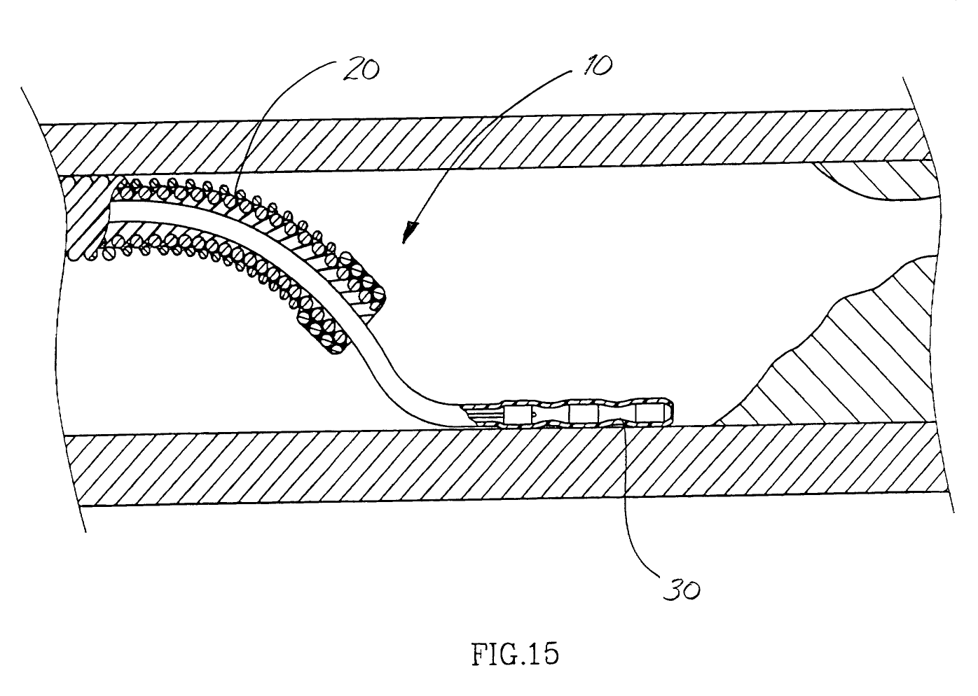

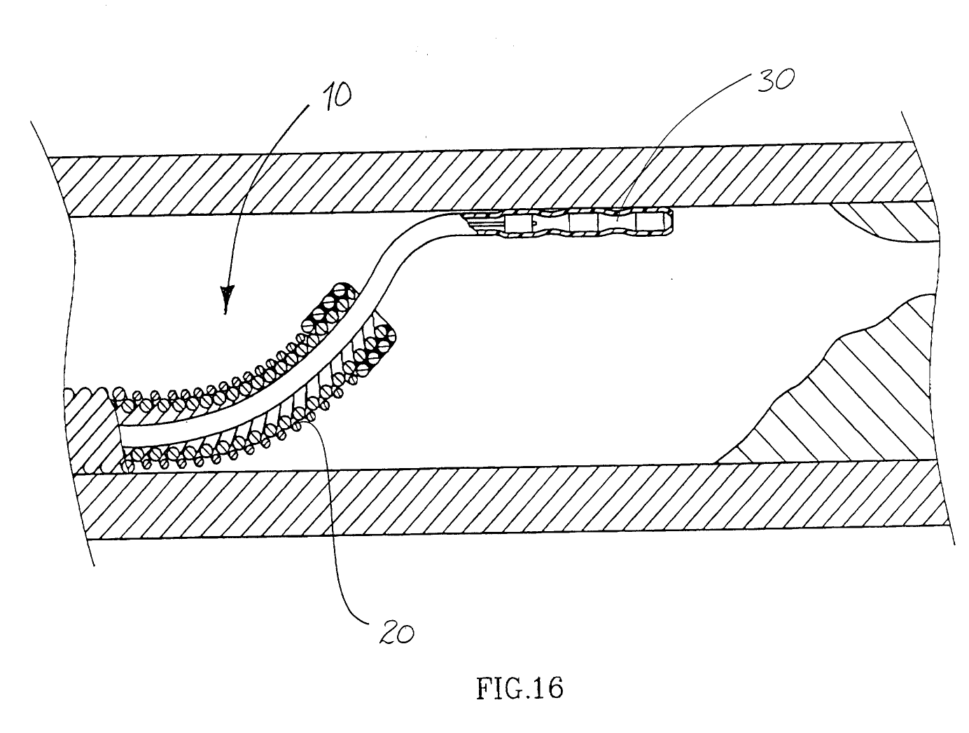

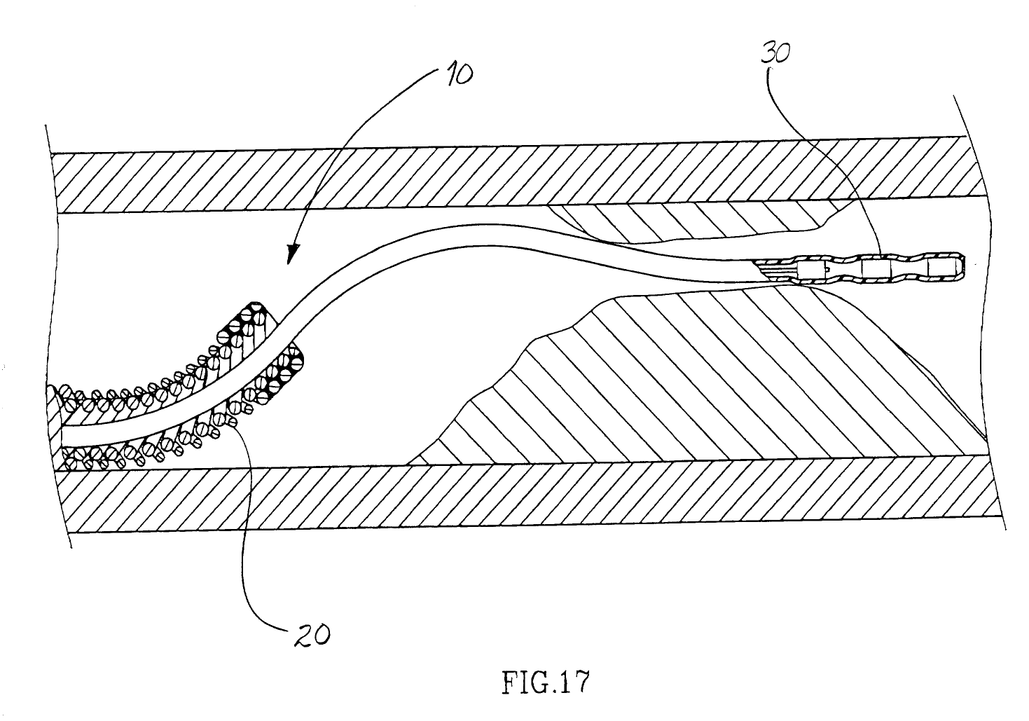

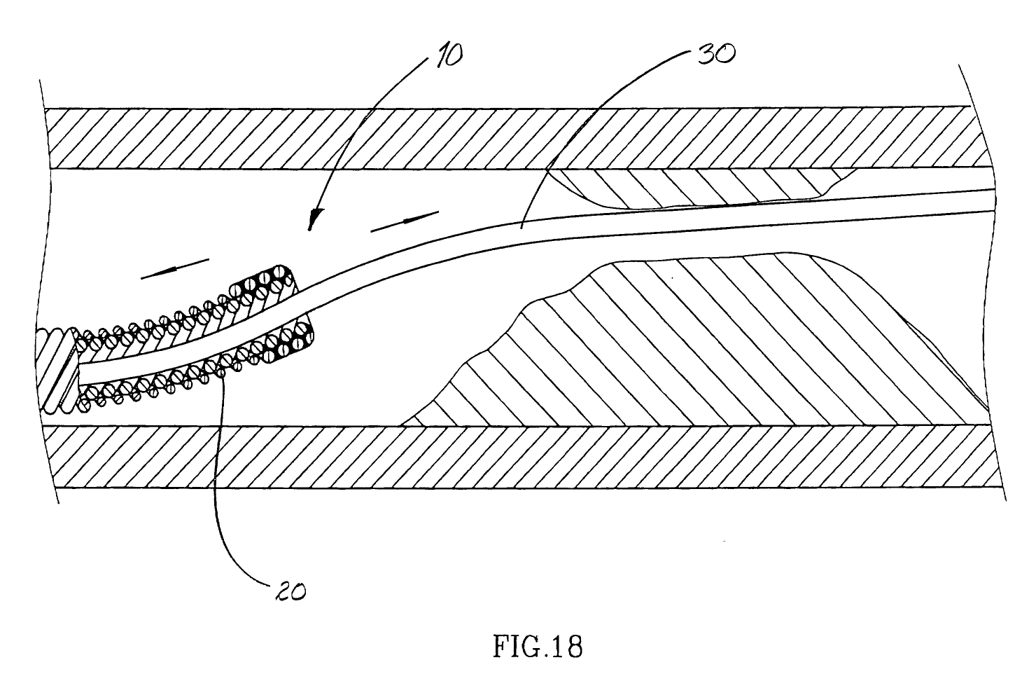

Referring now to FIGS. 13 through 18, there is illustrated, in sequential order, the operative steps employed to navigate the intravascular guidewire system of the subject invention within a blood vessel containing an eccentric stenotic lesion. Initially, as shown in FIG. 13, upon approaching the site of the lesion, the flexible distal end portion of the core wire is disposed within the normally curved distal section of the torquing sheath. Thus, the distal section is in its normally unstressed curved configuration in which a maximum degree of torque can be applied. Once at the site of the lesion, the flexible distal portion of the core wire is advanced from the distal end of the torquing sheath as shown in FIG. 14, and positioned against the interior wall of the blood vessel, as shown in FIG. 15. Thereafter, as shown in FIG. 16, the torquing sheath 20 and core wire 30 are easily rotated 180 to enable a more effective approach angle for the core wire to traverse the eccentric lesion. Then, as shown in FIG. 17, the core wire 30 is advanced through the lesion, and the torquing sheath 20 is withdrawn from the site of the lesion as shown in FIG. 18, whereby the core wire remains in an operational position within the blood vessel.

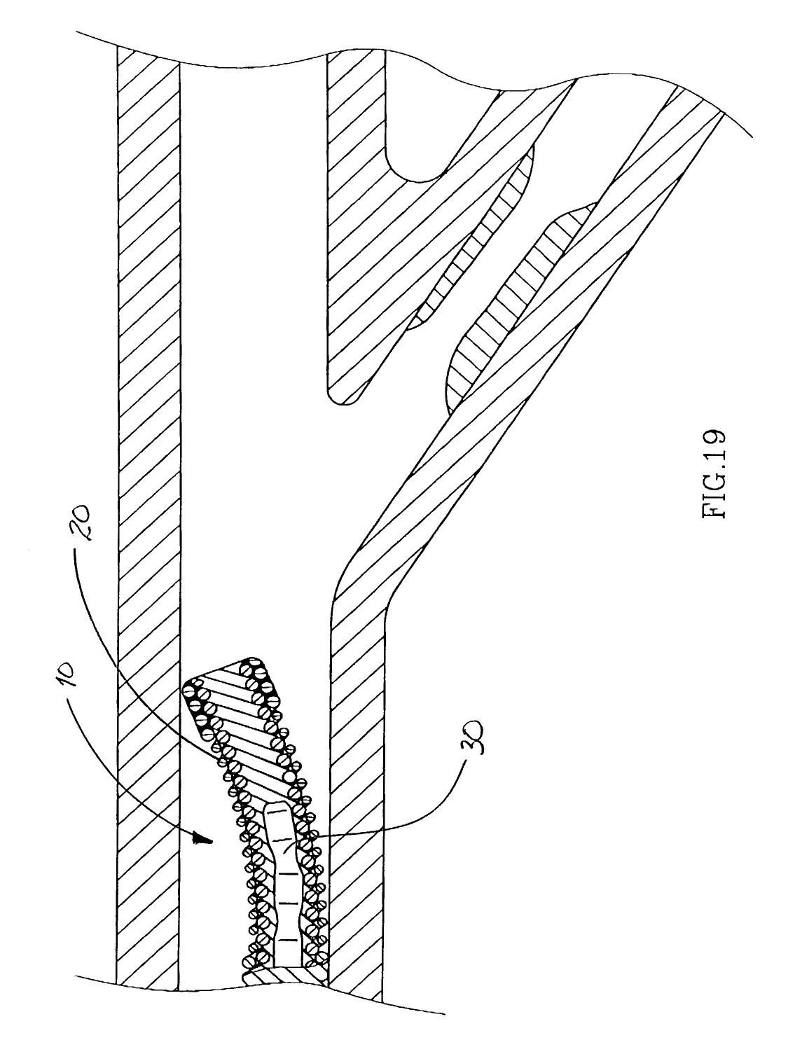

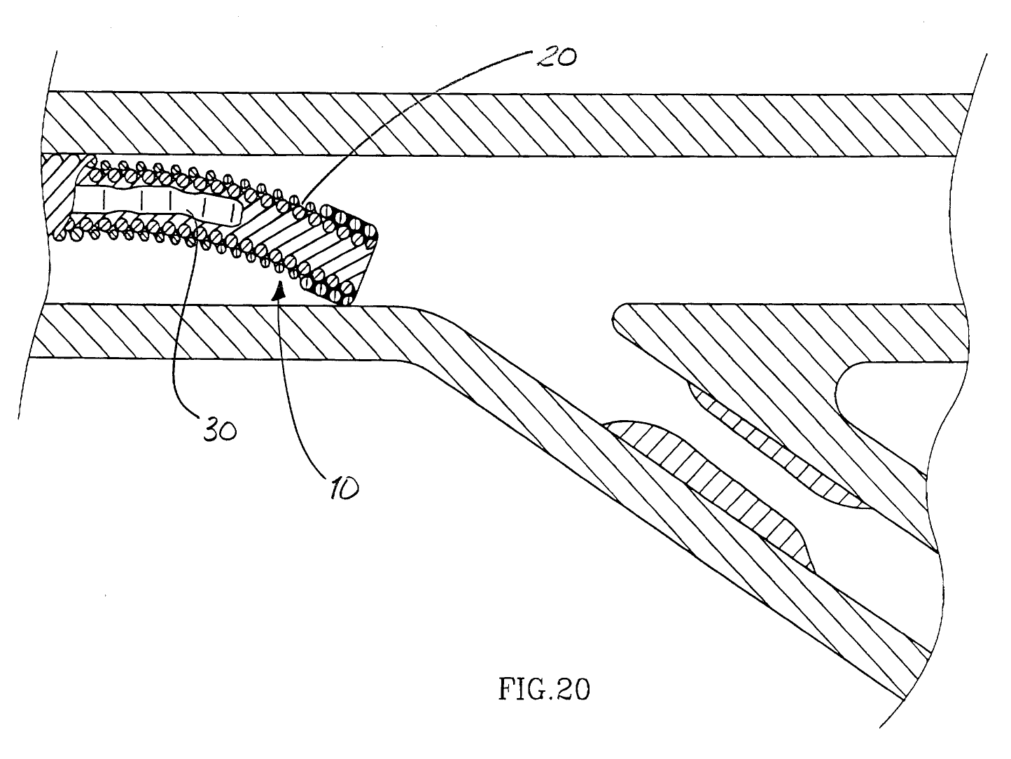

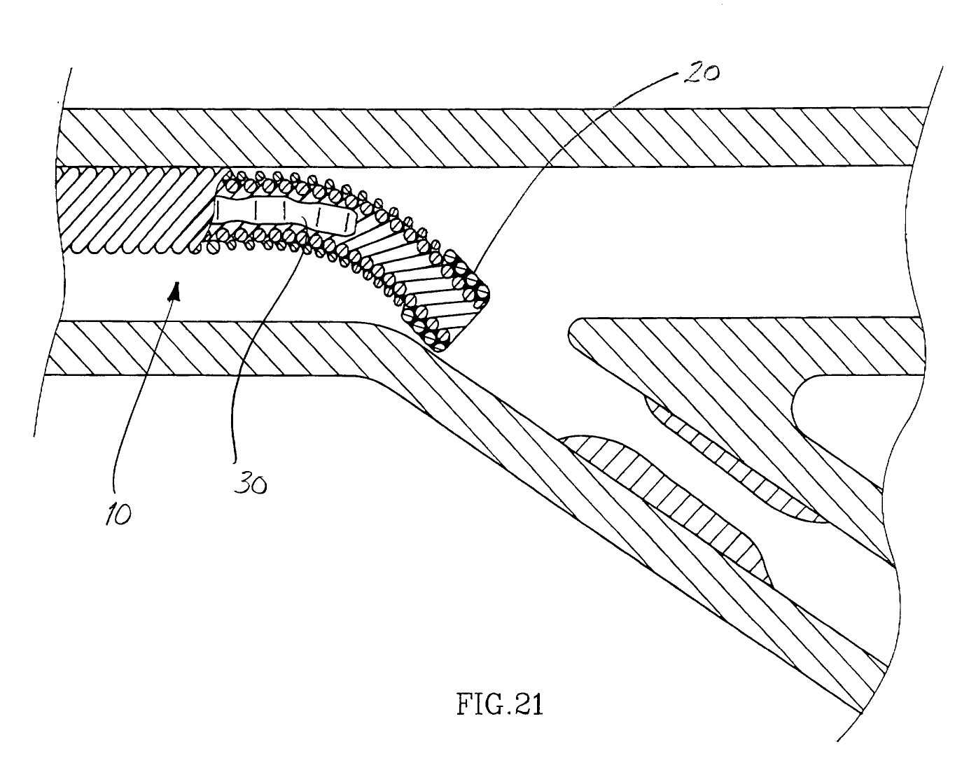

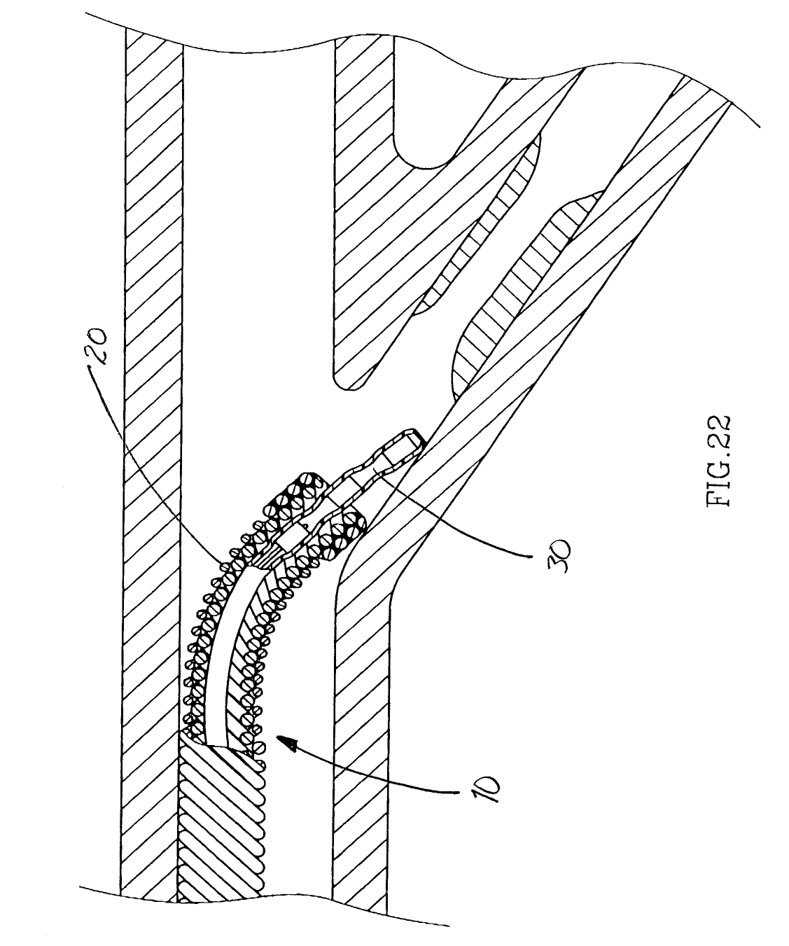

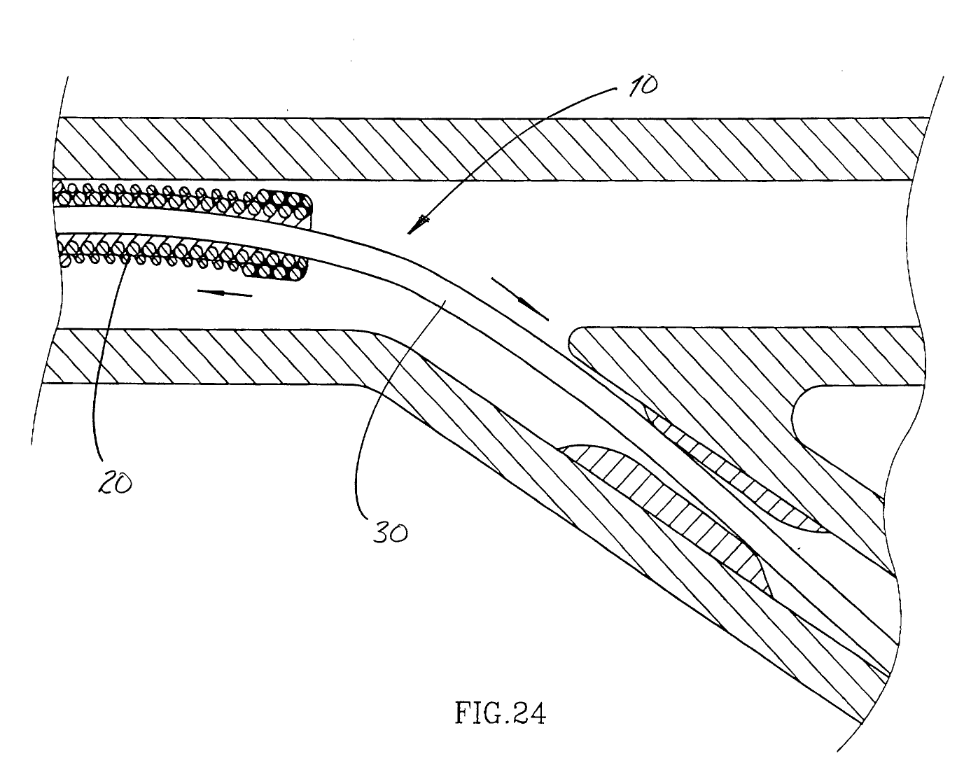

Referring in sequential order to FIGS. 19 through 24, there is illustrated a series of steps depicting the navigation of the intravascular guidewire system 10 of the subject invention within the venous system of a patient in such a manner so as to gain forward entry into a branch vessel containing an eccentric stenotic lesion. Initially, as shown, in FIG. 19, the normally curved distal section, of the torquing sheath 20 approaches the opening to the branch vessel distally. At such a time, the flexible distal portion of the core wire 30 is partially retracted within the sheath so that it has no effect on the curvature of the sheath. Then, as shown in FIG. 20, the normally curved distal section of torquing sheath 20 is rotated 180 degrees relative to the orientation shown in FIG. 19 to enable a more effective approach angle into the opening of the branch vessel. Thereafter, the curved distal section of torquing sheath 20 is advanced into the opening of the branch vessel, as illustrated in FIG. 21.

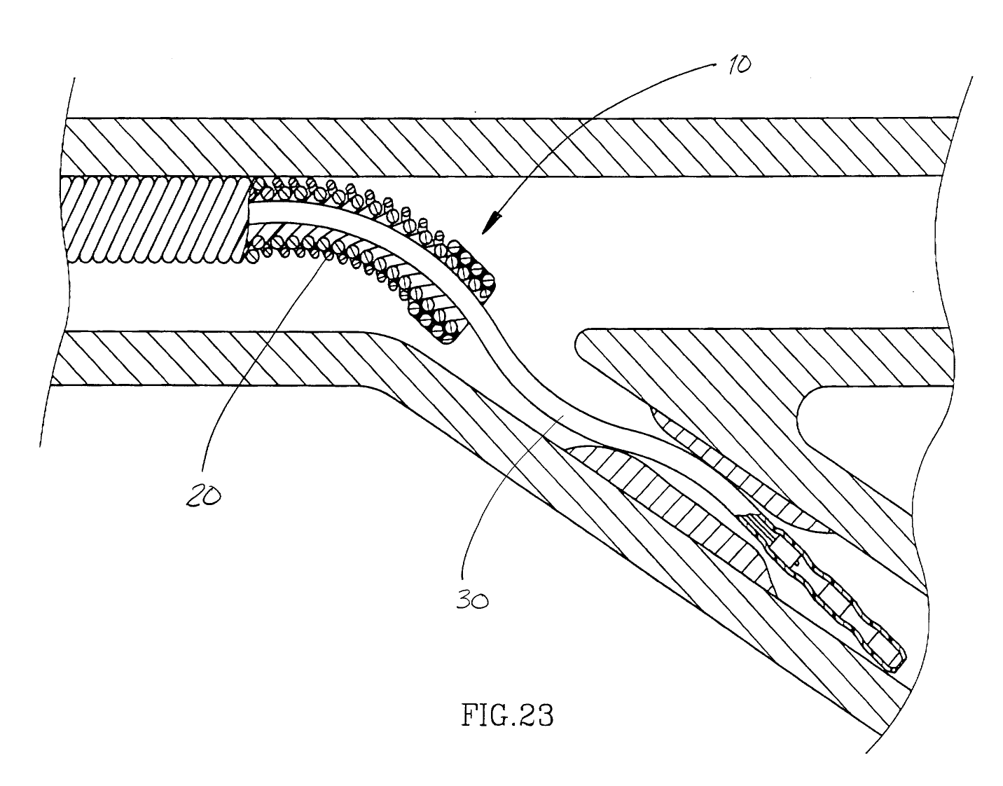

Up until this time, the distal portion of core wire 30 remains retracted within the lumen of the sheath 20. However, shortly thereafter, the core wire 30 is extended from the distal end of the sheath as shown in FIG. 22. and advanced into the branch vessel. Continued advancement of the core wire into the branch vessel and through the eccentric lesion, as depicted in FIG. 23, cause the curved distal section of sheath 20 to deflect from its normally curved state as the more rigid section of the core wire 30 interacts therewith. Once the core wire 30 has been advanced a sufficient distance through the lesion, the sheath is withdrawn from the blood vessel leaving a low profile core wire 30 behind. At such a time, the rigid portions of the core wire 30 interact with the sheath 20 so as to cause the curved distal section thereof to deflect into a relatively straight configuration. This enables the sheath to be withdrawn with relative ease. With the low-profile core wire 30 in place relative to the lesion, an intravascular device such as a rotational ablation device or balloon catheterization device may be directed to the lesion over the core wire 30.

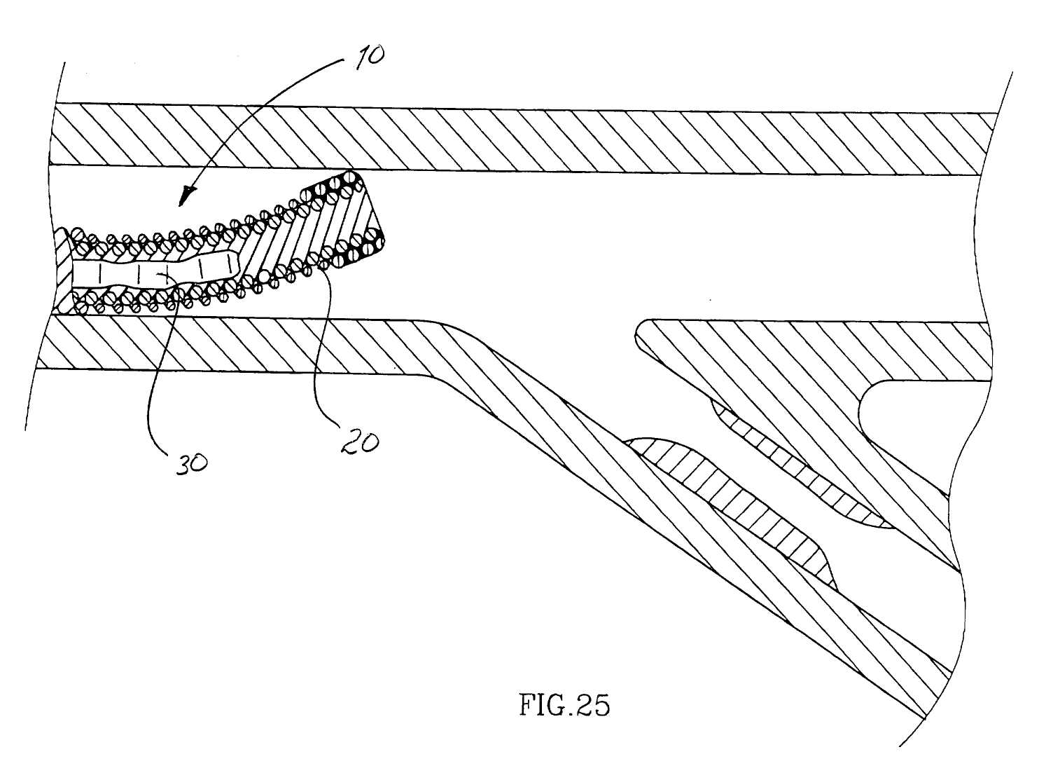

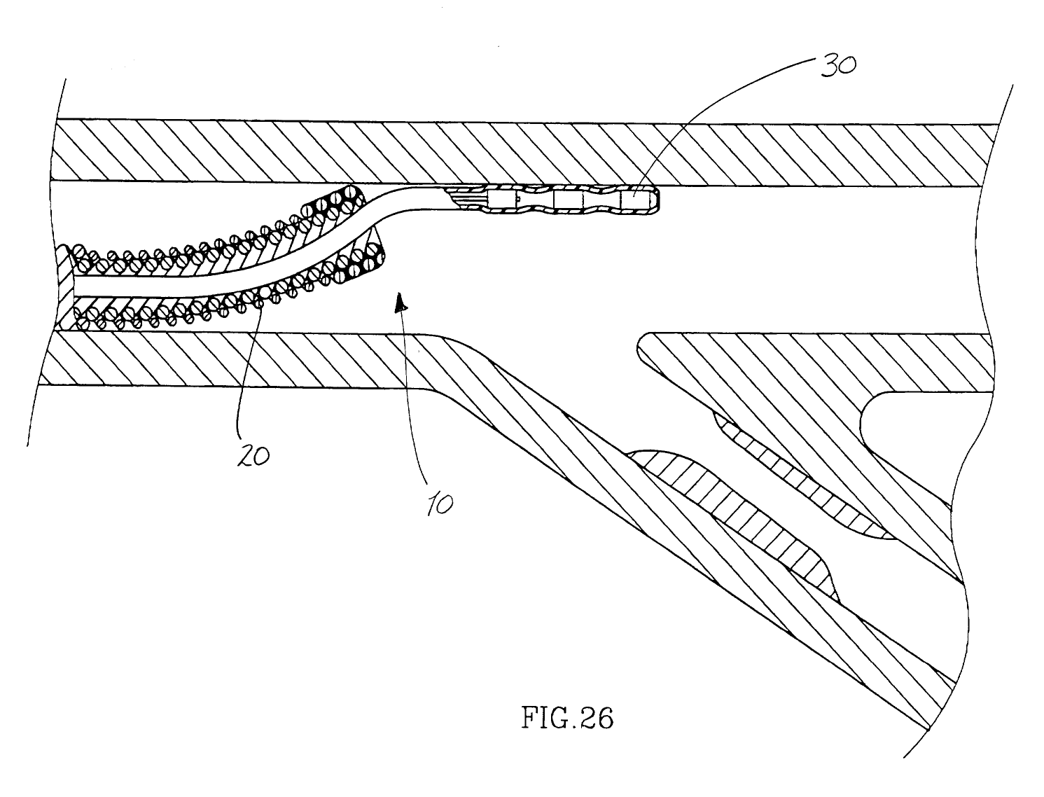

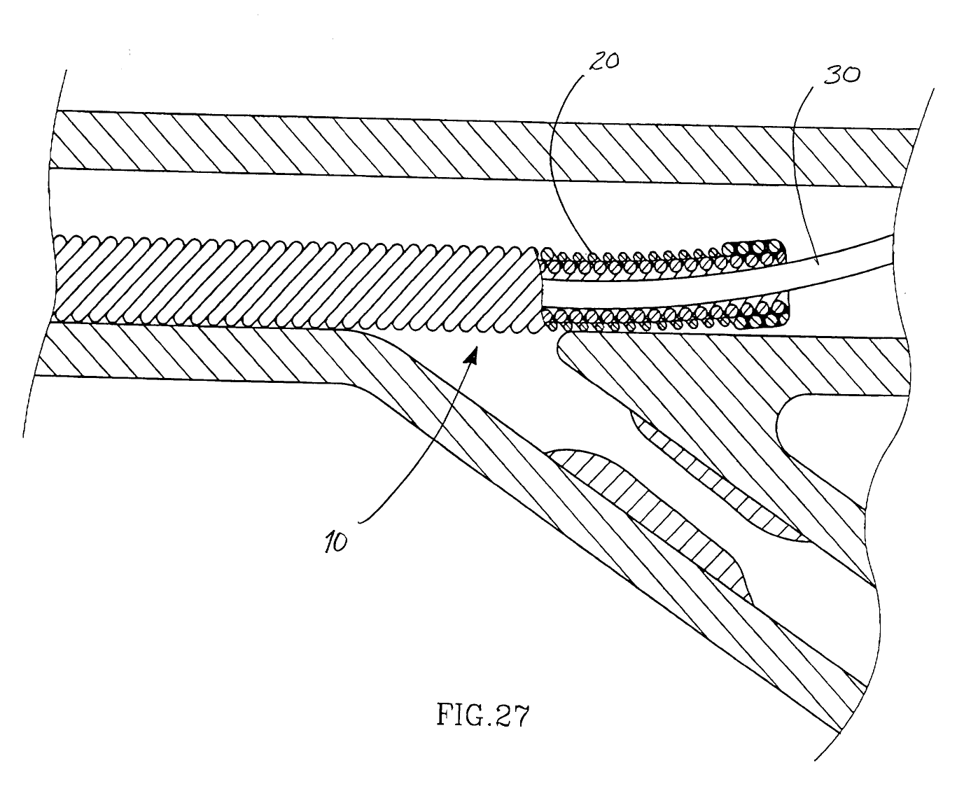

Referring is sequential order to FIGS. 25 through 32, there is illustrated a series of steps similar to those described above which depict the navigation of the intravascular guidewire system 10 of the subject invention within the venous system of a patient in such a manner so as to gain a rearward entry route into a branch vessel containing an eccentric stenotic lesion. As in the forward entry procedure described above, FIG. 25 illustrates an initial orientation of the normally curved distal section of the torquing sheath 20 as it approaches the opening to the branch vessel. At an appropriate location, the distal section of the sheath is rotated 180 degrees and the core wire 30 is advanced from the distal end thereof a sufficient distance so as to traverse the opening to the branch vessel, as shown in FIG. 26. At such a time, a relatively rigid portion of the core wire interacts with the normally curved distal section of the sheath causing it to deflect into a straightened condition. Then, as shown in FIG. 27, the distal section of the torquing sheath 20 is advanced over the core wire 30, to a position that is proximal to the branch vessel.

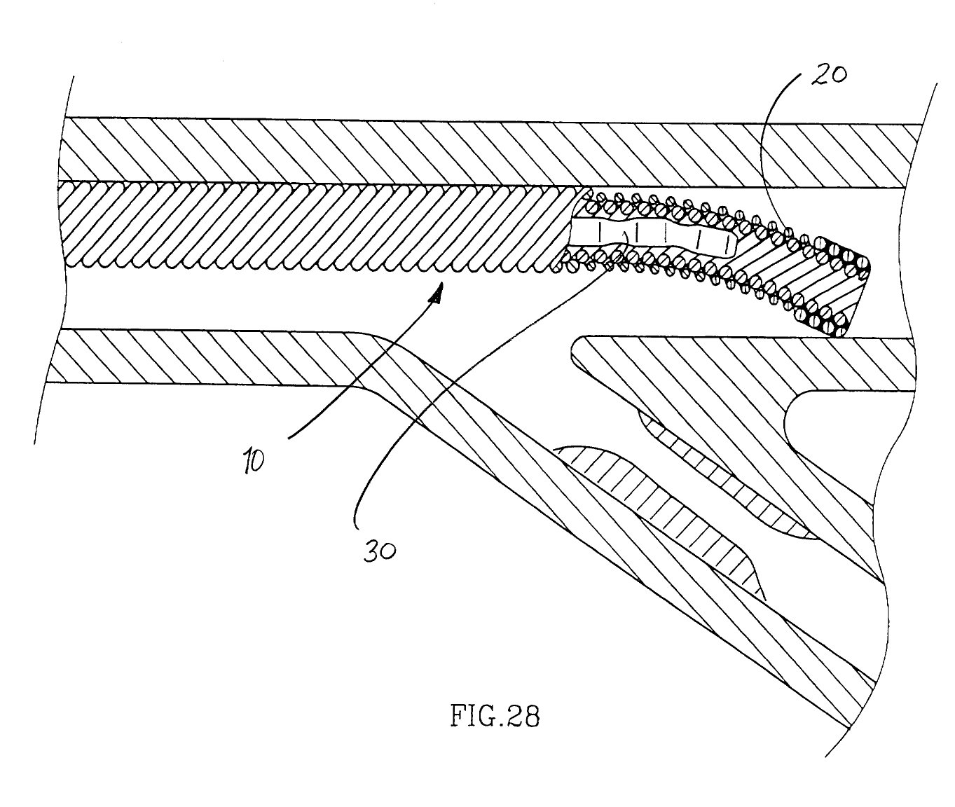

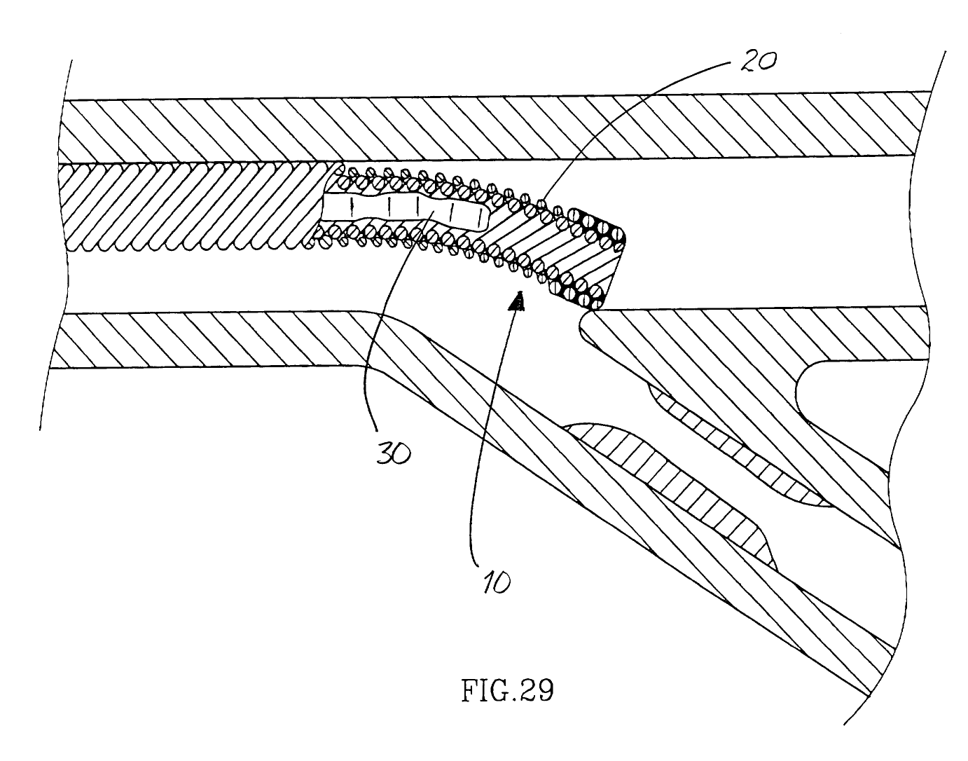

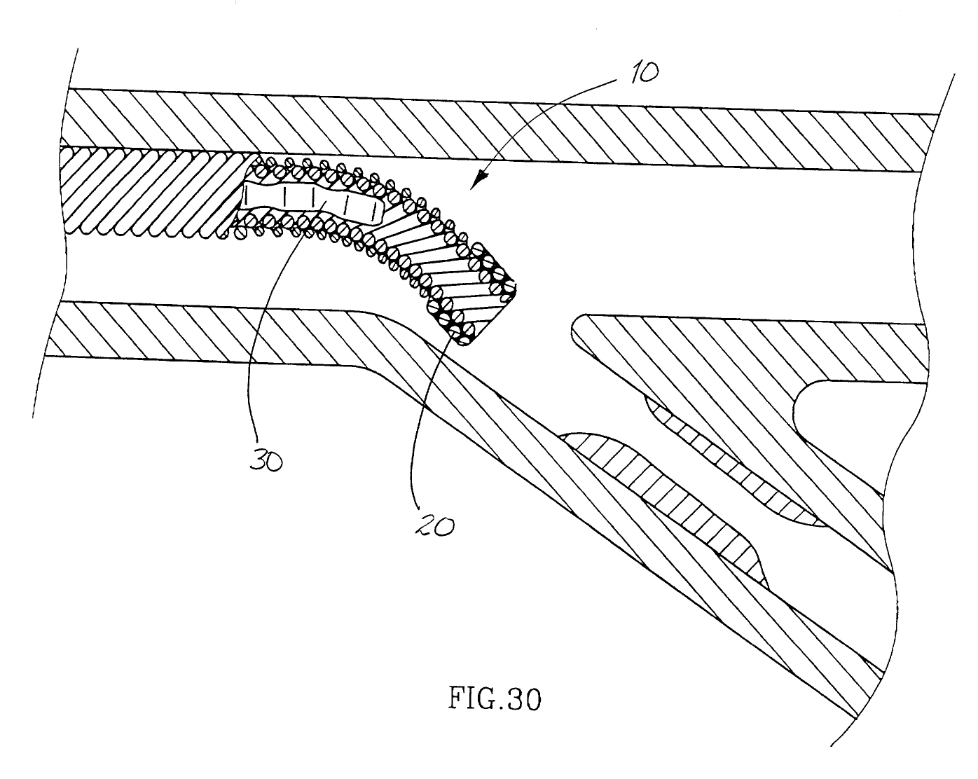

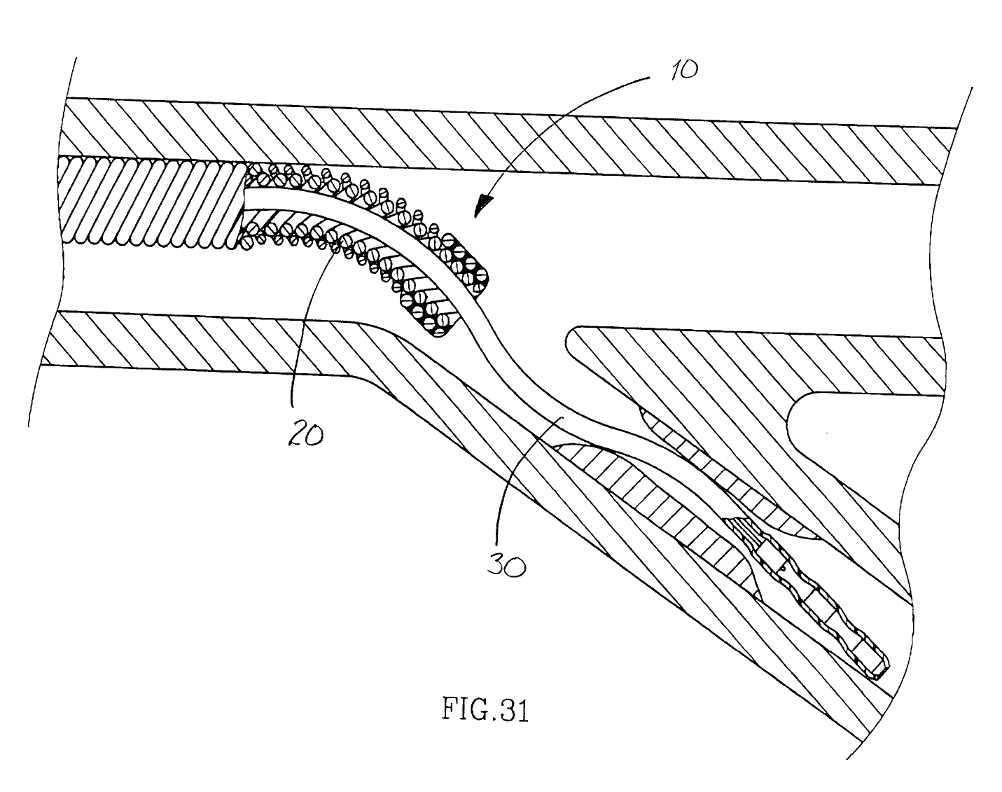

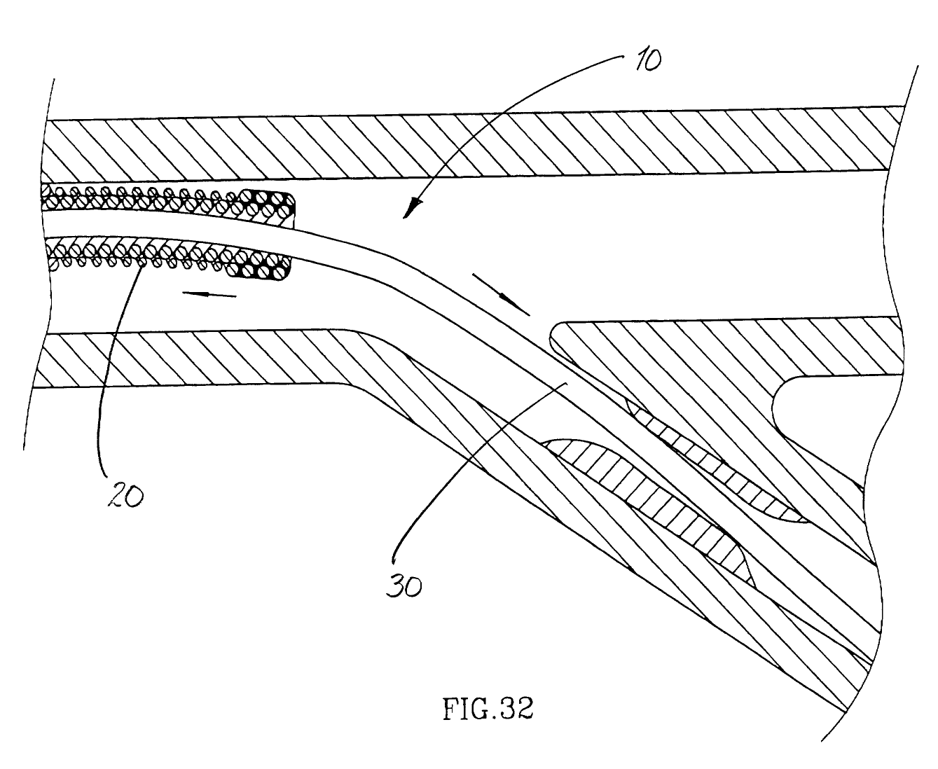

Thereafter, as best seen in FIG. 28, the core wire 30 is withdrawn into the lumen of torquing sheath 20 a sufficient distance so that the distal section of the torquing sheath is permitted to return to its normally curved configuration. The curved distal section of torquing sheath 20 is then moved proximally so that is it approaches the opening of the branch vessel in a rearwardly directed manner, as shown in FIG. 29. Proximal movement of the torquing sheath continues until the curved distal section of the sheath projects into the opening of the branch vessel, as best seen in FIG. 30. Thereafter, as illustrated in FIG. 31, the core wire 30 is advanced from the distal end of the torquing sheath into the branch vessel and through the lesion contained therein. During this advancement, the relatively rigid portions of the core wire 30 interact with the normally curved distal section of the sheath 20, causing the distal section to deflect into a more straightened condition. Thus, when the sheath 20 is withdrawn from the site of the lesion as shown in FIG. 32, there is little if any resistance to such movement.

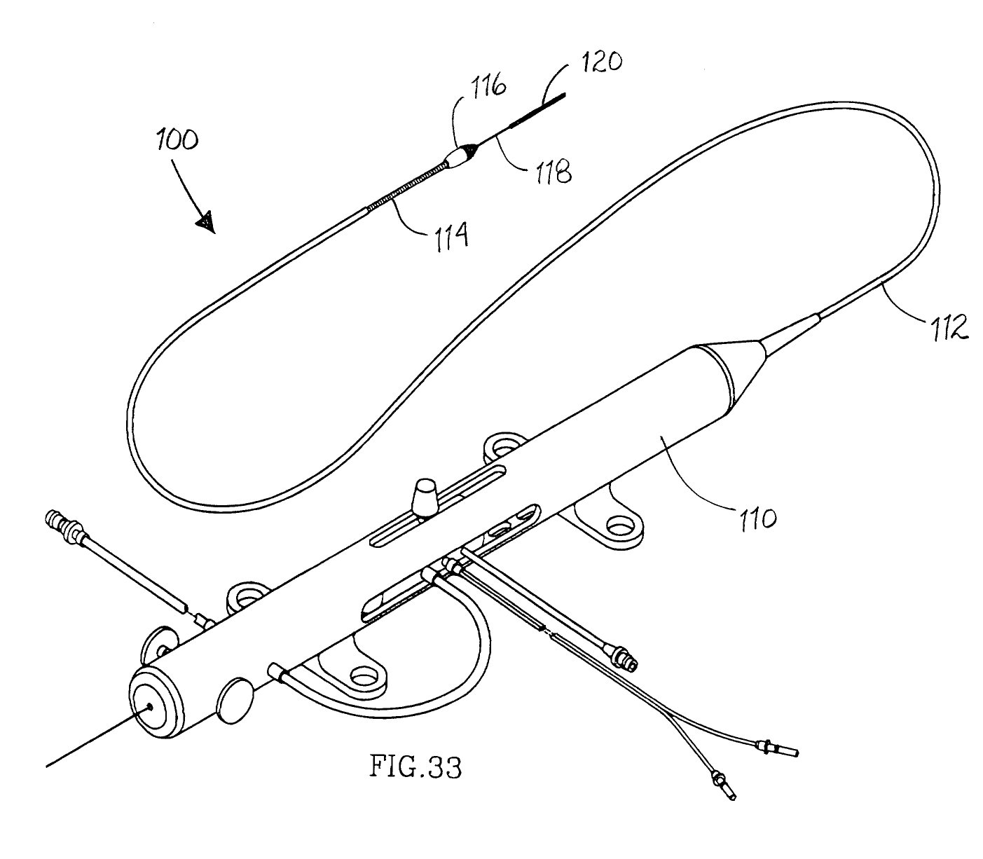

Referring now FIG. 33, there is illustrated a prior art rotational ablation system, manufactured by Boston scientific Corporation, and sold under the tradename Rotoblator®, designated generally by reference numeral 100. Ablation system 100 includes a handheld advancing device 110. An elongated tubular sheath 112 extend from the distal end of the advancing device 110. A flexible drive shaft 114 extend through tubular sheath 112 and has a diamond coated burr 116 supported at the distal end thereof. A flexible guidewire 118 extends through an interior lumen of drive shaft 114 for guiding the navigation of the drive shaft and burr through the venous system of a patient. Guidewire 118 is of the type which has a flexible spring tip 120 soldered, welded or otherwise secured to the distal end of the wire to render the distal end of the wire generally atraumatic.

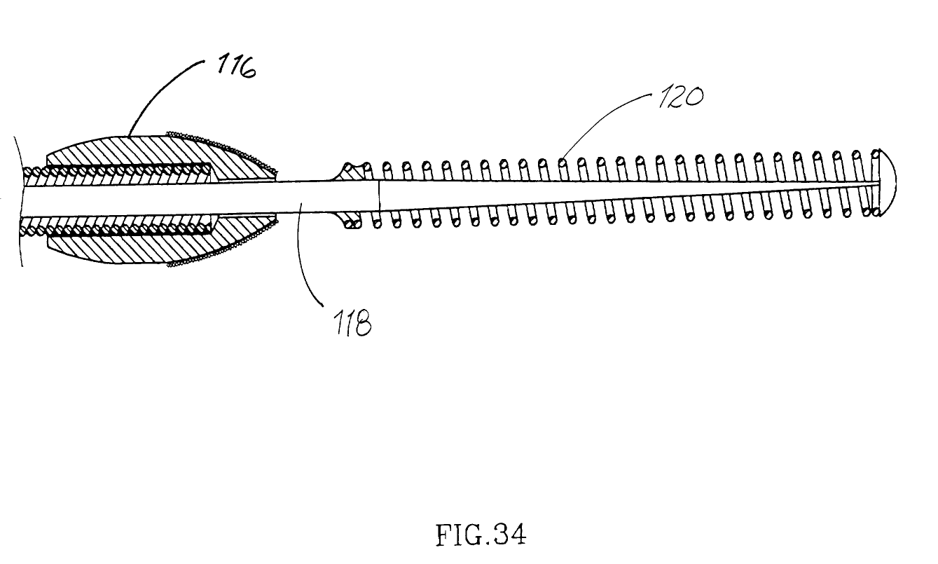

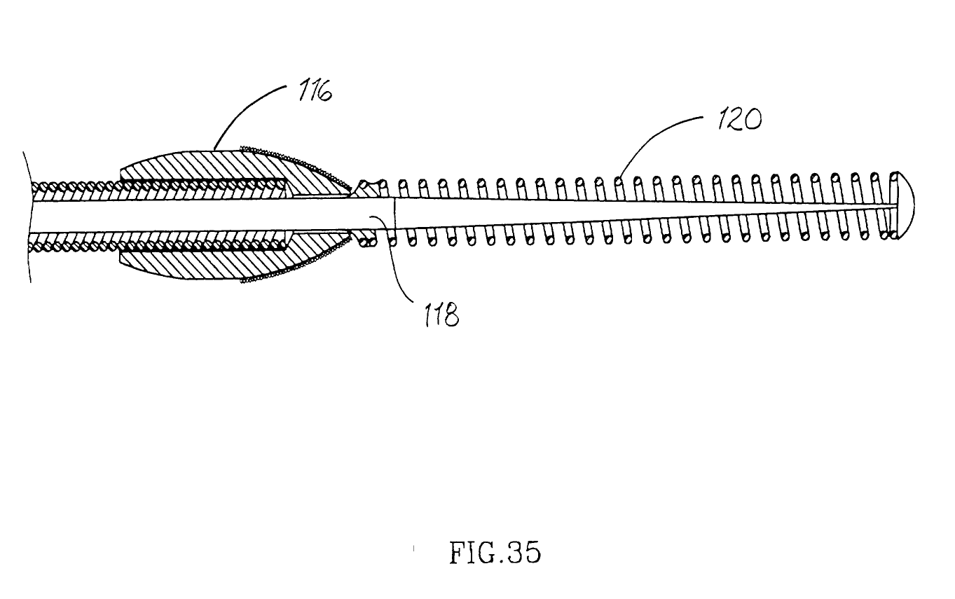

In use, burr 116 rotates at an extremely high rate of speed of between 140,000 to 180,000 rpm, and is oscillated back and forth over guidewire 118 between a proximal position as shown in FIG. 34 and a distal position as shown in FIG. 35, as it is advanced through a stenotic lesion. As best seen in FIG. 35, when the abrasive surface of the burr 116 is in a distal-most position, it is in lose proximity to the fixation point of the safety spring 120. With the burr rotating at such high speed, there is a chance that the abrasive surface could contact the solder securing the spring 120 to the wire, causing the spring to dislodge from the guidewire. This would present a serious problem and require extreme measures for retrieval. Therefore, extreme care must be taken when using the prior art ablation device 100 to ensure that the burr 116 does not come into contact with the safety spring 120.

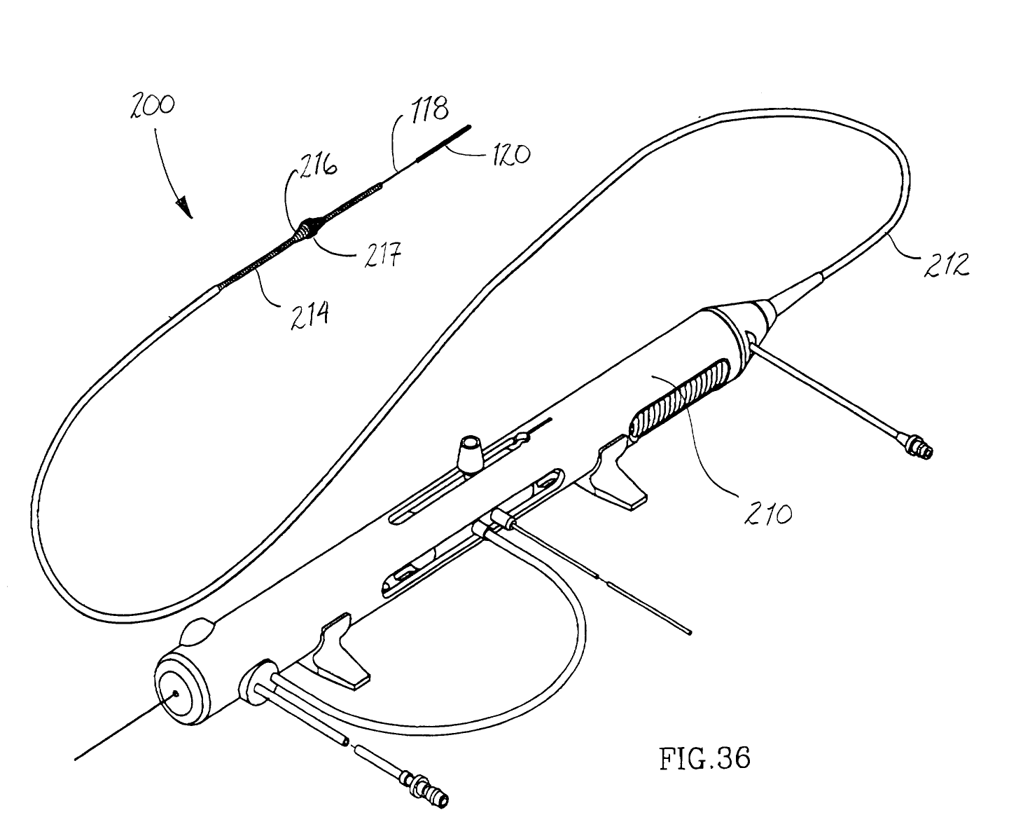

Referring now to FIG. 36, there is illustrated a rotational atherectomy device 200 as disclosed in an Provisional Application filed Oct. 19, 2001 entitled “Rotational Angioplasty Device With Abrasive Crown” and similar to that which is disclosed in U.S. Pat. No. 6,132,444 to Shturman, the disclosures of which are incorporated herein by reference in their entireties. In brief, atherectomy device 200 includes an advancing device 210 from which extends an elongated tubular sheath 212. A flexible drive shaft 214 formed from a helically wound coil extends through tubular sheath 112 and has an enlarged eccentric coil segment 216 formed thereon at a location that is spaced from the distal end of the shaft. A ring 217 extends about the outer periphery of the eccentric coil segment 216 which is has a diamond coated abrasive surface deposited thereon.

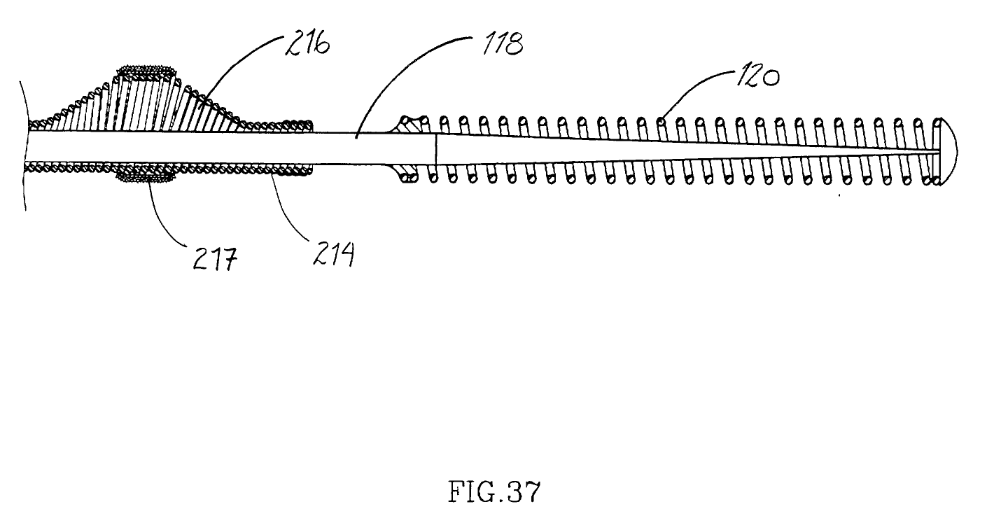

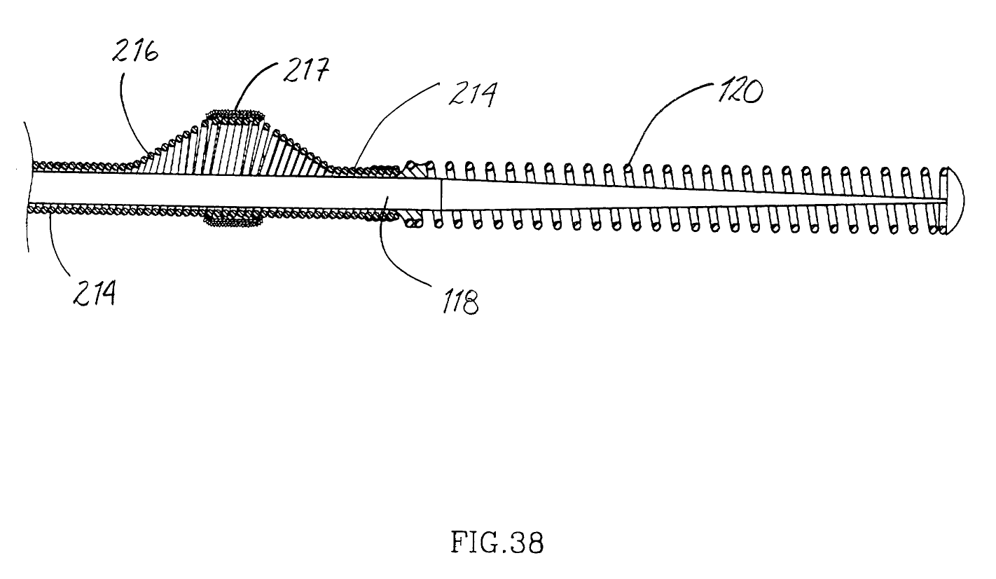

As illustrated in FIGS. 37 through 38, flexible guidewire 118 extends through an interior lumen of drive shaft 214 for guiding the navigation of the drive shaft through the venous system of a patient. As noted above, guidewire 118 is of the type which has a flexible spring tip 120 soldered to the distal end of the wire to render the distal end of the wire generally atraumatic. Unlike burr 116 however, the abrasive surface of eccentric coil segment 216 does not come into contact with the solder that secures the safety spring to 120 the guide wire 118 as it is oscillated between the proximal position of FIG. 37 and the distal position of FIG. 38. Nevertheless, if the distal portion of the drive shaft 214 contacts the solder connection, it too could dislodge the safety spring 120.

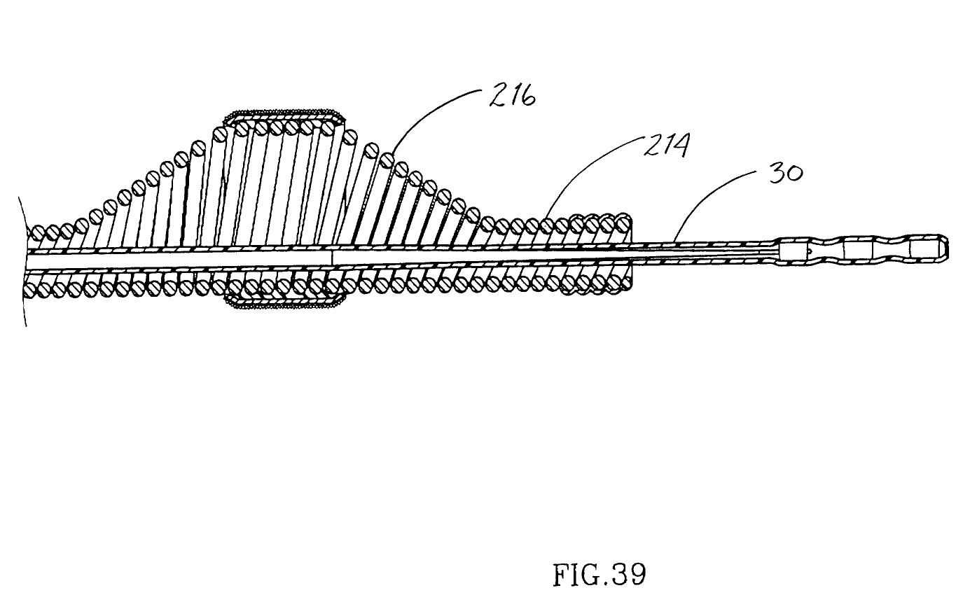

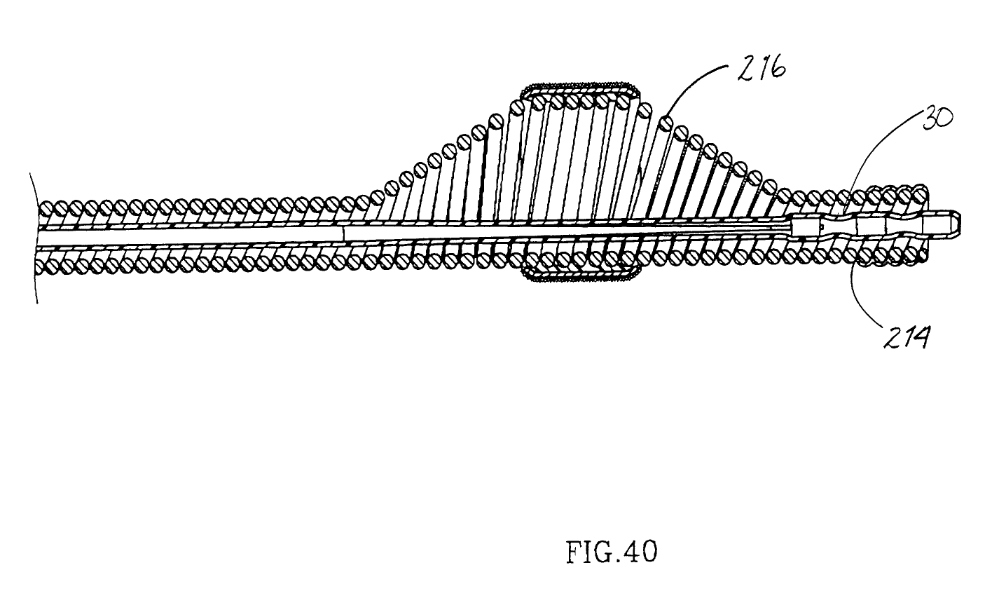

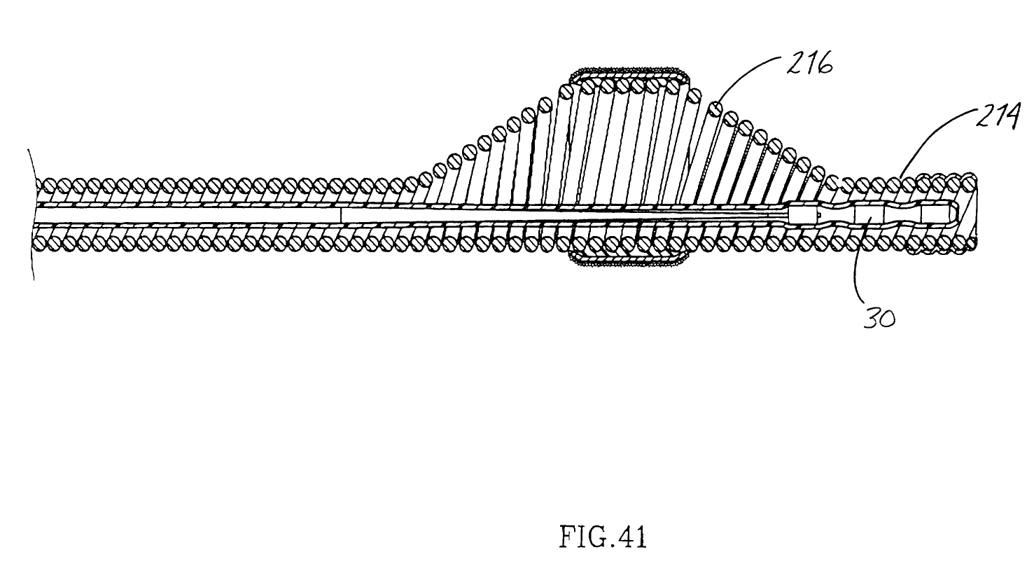

Referring to FIGS. 39-41, wherein the core wire 30 of the subject invention is utilized in conjunction with the rotational atherectomy device 200 rather than the prior art guidewire 118. With such an arrangement, the drive shaft 214 can be safely and advantageously moved along the entire length of the core wire without risk. More particularly, the drive shaft 214 can be moved from a proximal position as shown in FIG. 39 wherein the crown of the eccentric coil segment 216 is disposed in the transitional region of core wire 30, to a more distal location as shown in FIG. 40 wherein the crown of the eccentric coil segment 216 is located in the conically tapered region of core wire 30. In this position, the distal portion of drive shaft 214 surrounds the flexible distal portion of core wire 30. As shown in FIG. 41, the design and configuration of core wire 30 enables the drive shaft 214 to be advanced so far so as to allow the distal end of the drive shaft 214 to advantageously extend beyond the distal end of the core wire 30.

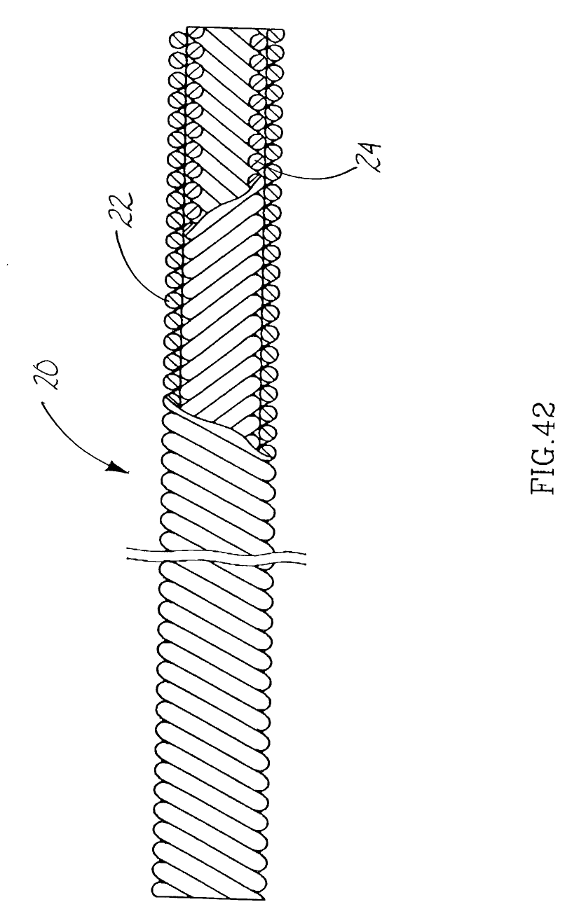

Referring now to FIG. 42, there is illustrated a distal portion of the two layer torquing sheath 20 of the subject invention prior to the application of any manufacturing processes thereon. At such a time, the opposed ends of the oppositely wound inner and outer coil layers 22, 24 of torquing sheath 20 have not yet been joined to one another. In accordance with certain preferred embodiments of the subject invention, the distal section of torquing sheath 20 is treated in either of two different manners to form a desired radius of curvature.

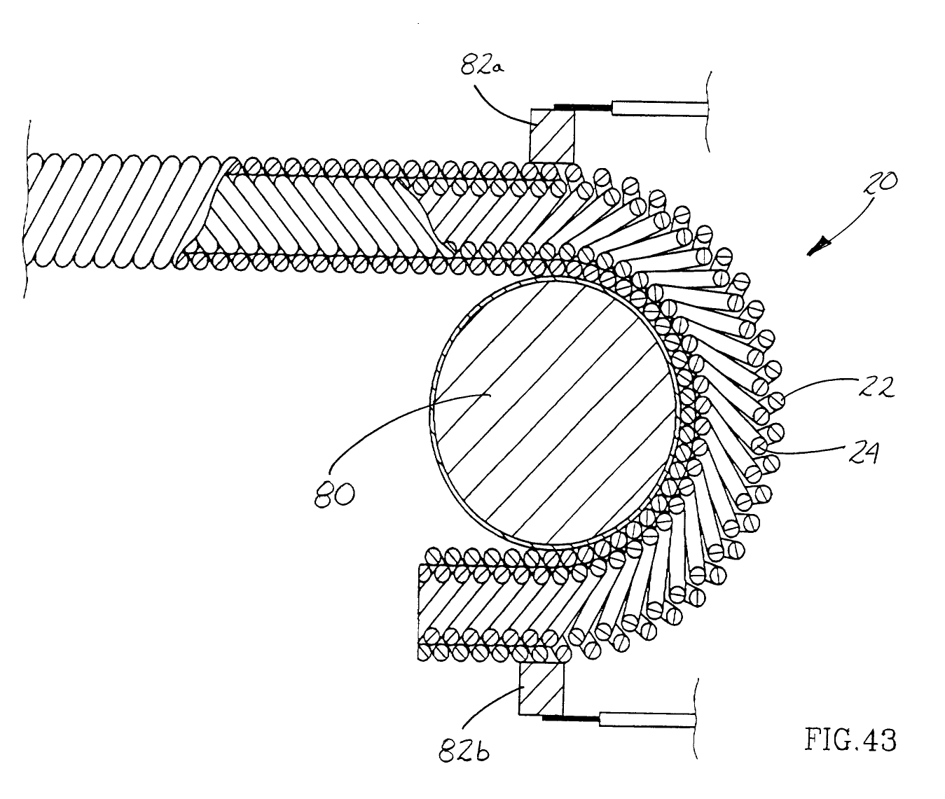

For example, as shown in FIG. 43, the curved distal section of torquing sheath 20 can be formed by first placing the distal section about a cylindrical aluminum forming mandrel 80 having an oxidized surface layer and a diameter corresponding to the desired radius of curvature. The aluminum oxide ensures that current will flow through the coils of the sheath, rather than simply across the mandrel. A suitable fixture (not shown) is employed to secure the sheath to the mandrel. Diametrically opposed contacts 82a and 82b are positioned against the outer coil layer 22. The contacts are electrically connected to a capacitor (not shown), which is discharged at an appropriate time so as to cause current to flow through the coils of the sheath. As a result, the distal portion of sheath 20 assumes the radius of curvature of mandrel 80.

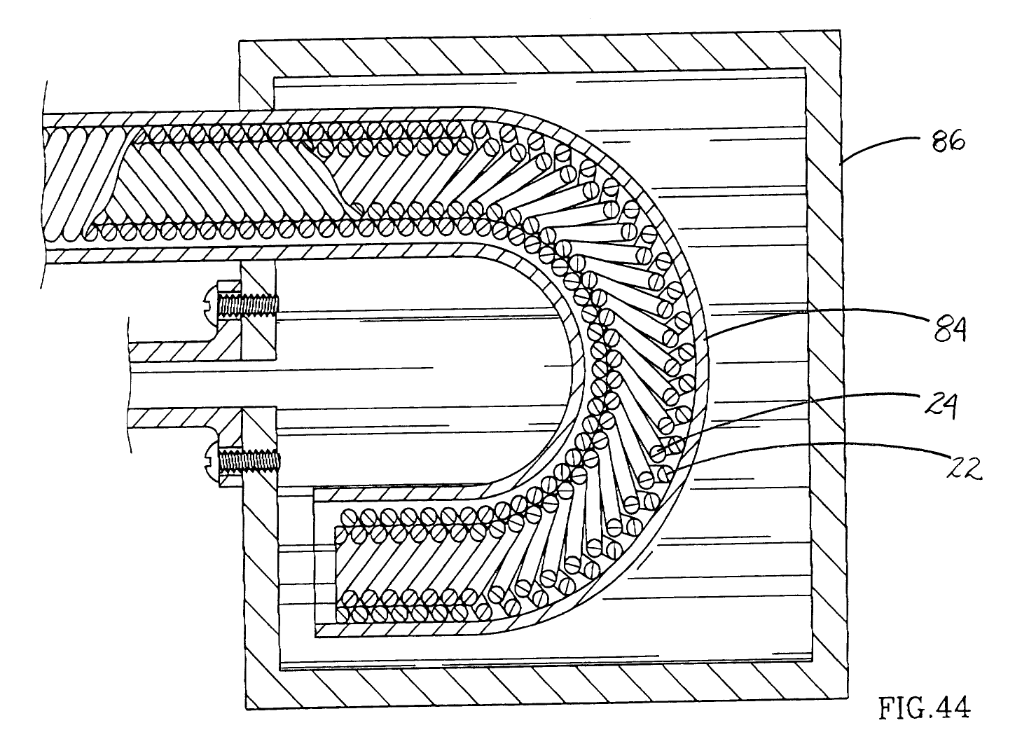

Alternatively, the curved distal section of torquing sheath 20 may be formed by heat treatment. More particularly, as illustrated in FIG. 44, the distal section of torquing sheath 20 is placed into a tubular metal sleeve 84 having the desired radius of curvature. The sleeve 84 is then placed into an oven 86 which is filled with an inert gas, preferably argon, and is heated at a sufficient temperature and for a sufficient time period so as to relieve the stress in the wire coils and enable the sheath to assume the desired curvature.

As illustrated in FIG. 45, after the distal section of the sheath has been treated to provide the desire radius of curvature, the inner and outer coil layers 22, 24 are secured to one another by way of soldering 25, welding or other methods known in the art. In the case of soldering, a medical grade silver or silver-based solder material is used. Once soldered, the inner and outer coil layers 22, 24 are coaxially stabilized in that they cannot shift relative to one another in an axial direction.

As noted above with reference to FIGS. 4 and 5, the curved distal section 20a as well as a portion proximal of the sheath 20 proximal thereto can have a reduced diameter to provide the distal section of the sheath with added flexibility and reduced rigidity, in cases where the radius of curvature is relatively tight. Otherwise, the distal section 20a of the sheath 20 is not treated in this manner, since it would have sufficient flexibility due to the greater radius of curvature. To reduce the diameter of the distal section, and more particularly to reduce the diameter of the outer coil layer 22, a chemical etching process is performed. Initially, as shown in FIG. 46, the distal section of the torquing sheath is masked to focus the activity of the etching agent on the outer coil layer. The goal being to facilitate the etching process from the outer surface of the sheath. In particular, a preformed TFE bead 90 is disposed within the interior lumen of the sheath to mask the inner coil layer 24, and silicon sealant beads 92 and 94 are deposited at the distal end of the sheath to mask the solder connection and at a proximal location to define the proximal limit of the etching. A suitable TFE bead is available from Zeus of Raritan, N.J. Alternatively, a PTFE tube 96 may be use instead of the proximal silicon sealant bead 94 or in addition thereto. After the torquing sheath has been properly masked, the distal section thereof is emersed in a bath of sulfuric acid, or a similar etching agent. It is envisioned that processing time can be increased by electrically charging the solution.

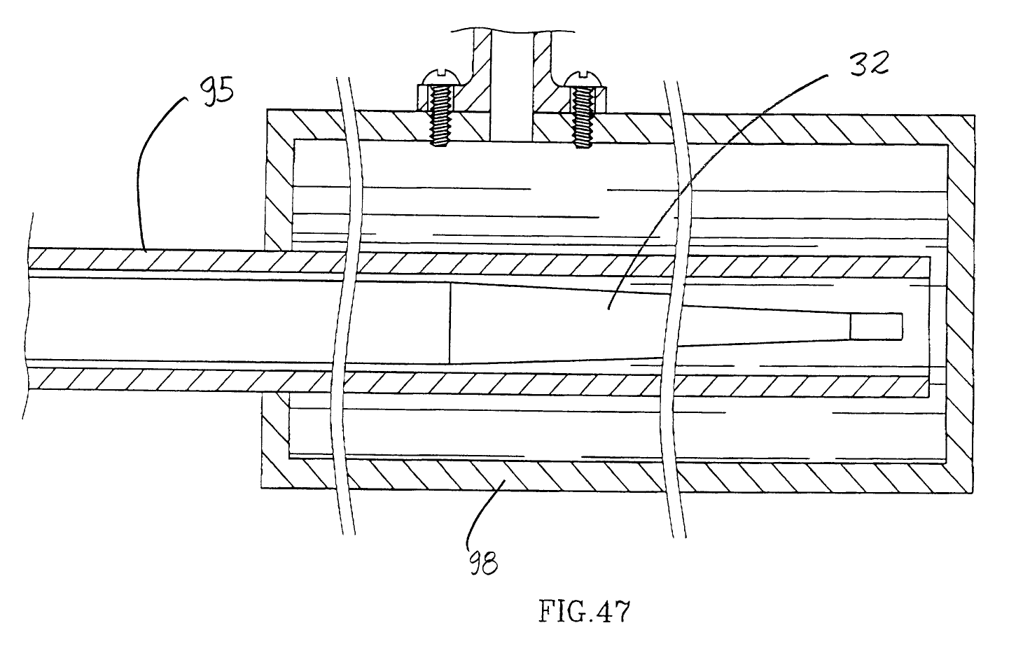

Referring now to FIG. 47, as discussed briefly hereinabove, the distal section of the wire body 32 of the core wire 30 (e.g., about 30 cm) is heat treated in such a manner so as to relieve or relax the super-elastic or shape memory characteristics of the alloy from which the wire is formed. This heat treatment in accomplished by positioning the distal portion of the wire body 32 into a tubular sleeve 95 which is extended into an oven 98. The oven is then filled with an inert gas, such as, argon. The distal portion of the wire body is then heated for about thirty (30) minutes at approximately 300° F. This treatment alters the metallurgic properties of the shape memory alloy in such a manner so as to remove the set that had previously been cast in the wire during its manufacture. Those skilled in the art will readily appreciate that this treatment is necessary if the core wire 30 is utilized for the guidance of rotation ablation devices as it serves to prevent fatigue failure in the wire body 32. However, if the core wire 30 is utilized in conjunction with a non-rotating device such as, for example, a balloon catheterization device, fatigue fractures are not an issue, and this process becomes unnecessary.

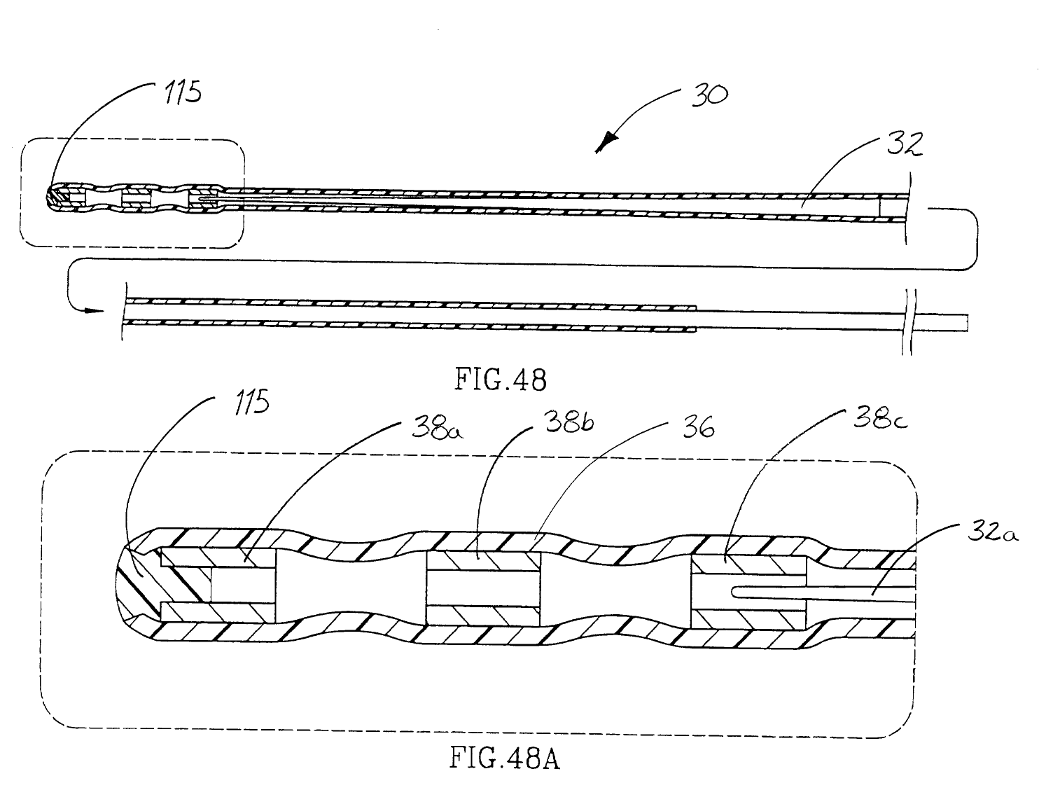

Referring now to FIGS. 48 and 48A, there is illustrated another embodiment of the core wire 30 of the subject invention wherein a plug of polymer material or epoxy 115 is positioned within the distal end of flexible sleeve 36 to prevent the possibility of the escape of air that could be trapped in sleeve 36. The plug 115 may be preformed or can consist of a sealant such as a medical grade glue or epoxy. In the absence of a plug, it is envisioned that contrast solution may be injected into sleeve 36 prior to insertion of the guide wire into a blood vessel. The solution would displace any air trapped within the sleeve 36 and enhance the radiopacity of the distal portion of the core wire.

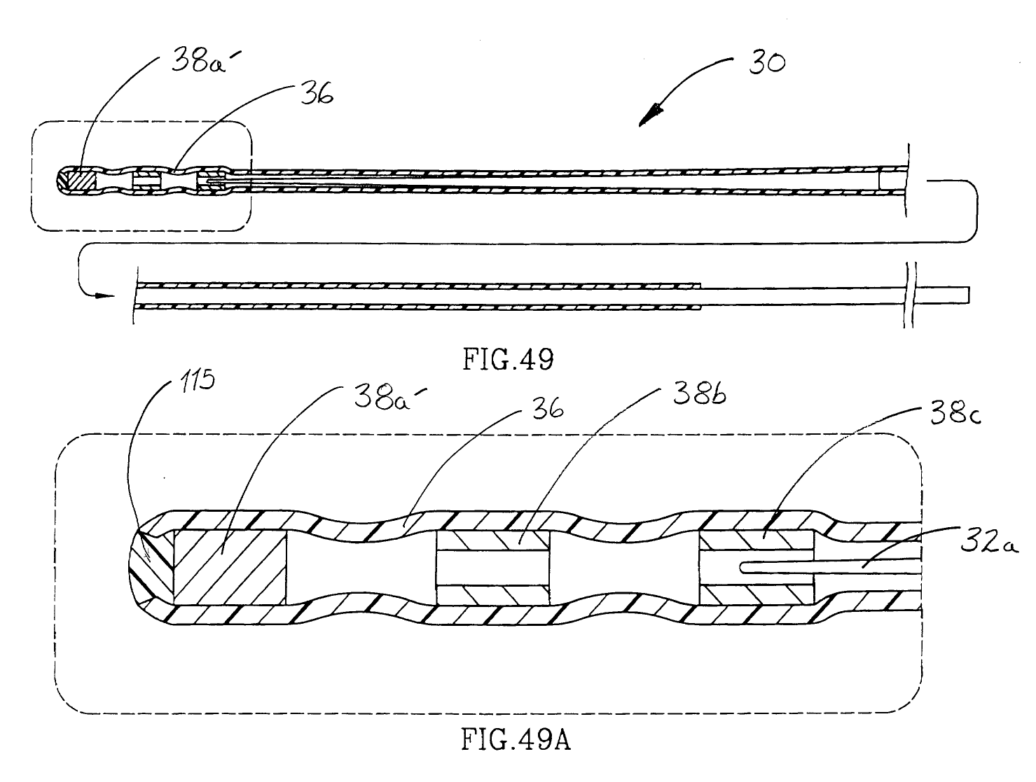

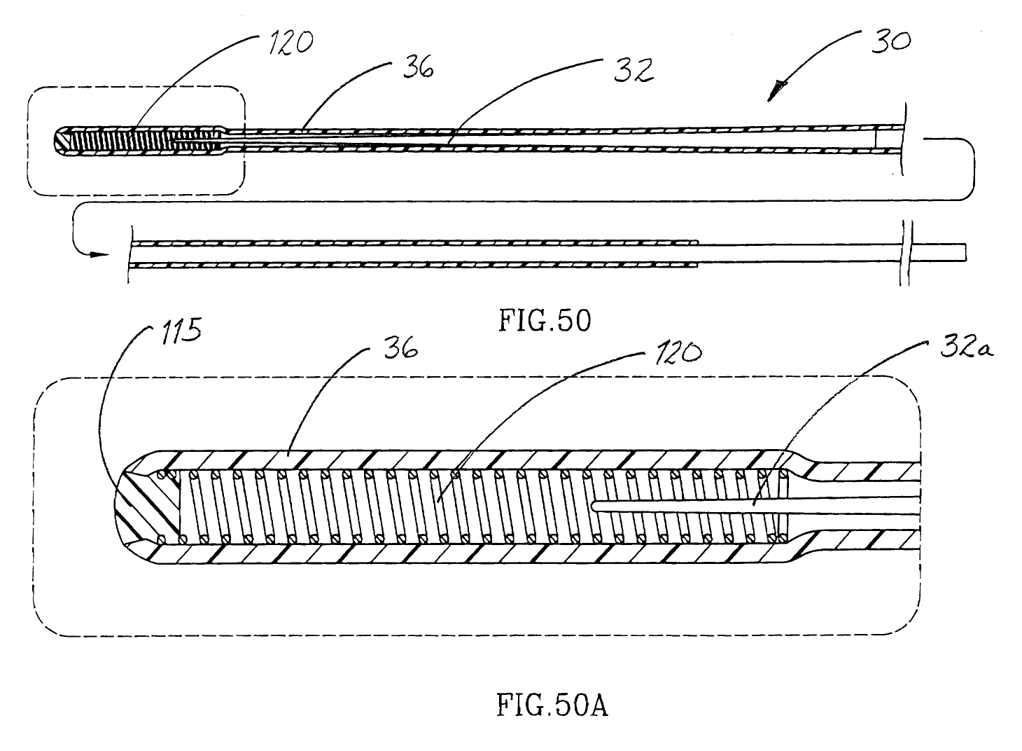

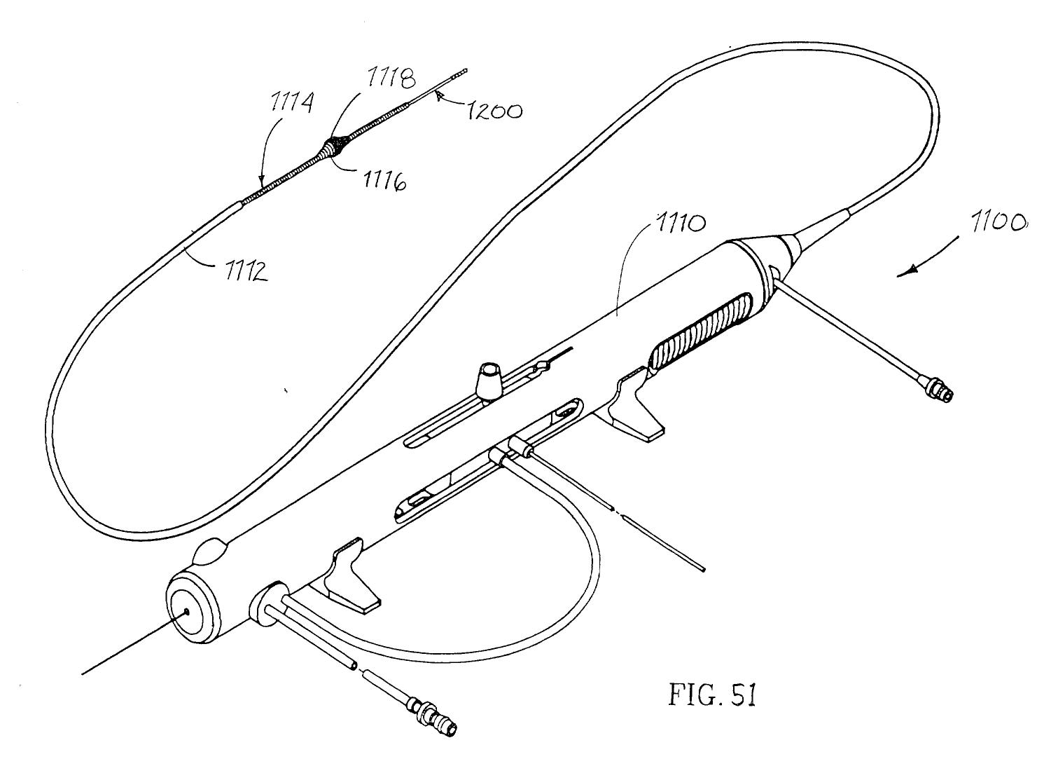

Referring to FIGS. 49 and 49A, there is illustrated another embodiment of the core wire 30 of the subject invention wherein the distal-most marker 38a in flexible sleeve 36 is defined by solid cylindrical member rather than a tubular member. This solid marker could be formed simply from a piece of radiopaque wire of suitable diameter. Once emplaced, the solid marker will prevent the egress of air from the sleeve or the ingress of bodily fluid into the sleeve. Referring to FIGS. 50 and 50A, there is illustrated yet another embodiment of the core wire 30 of the subject invention wherein a monofilar coiled marker wire is supported with the flexible tubing 36. The marker wire 120 is an alternative to the cylindrical markers 38a-38c described hereinabove, and provides enhanced flexibility.

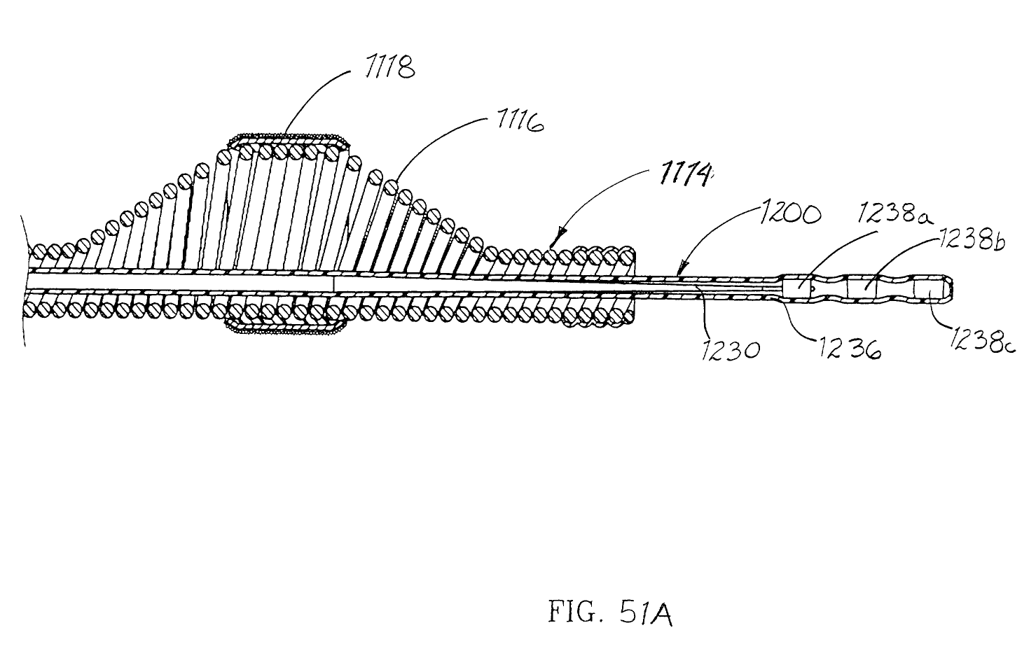

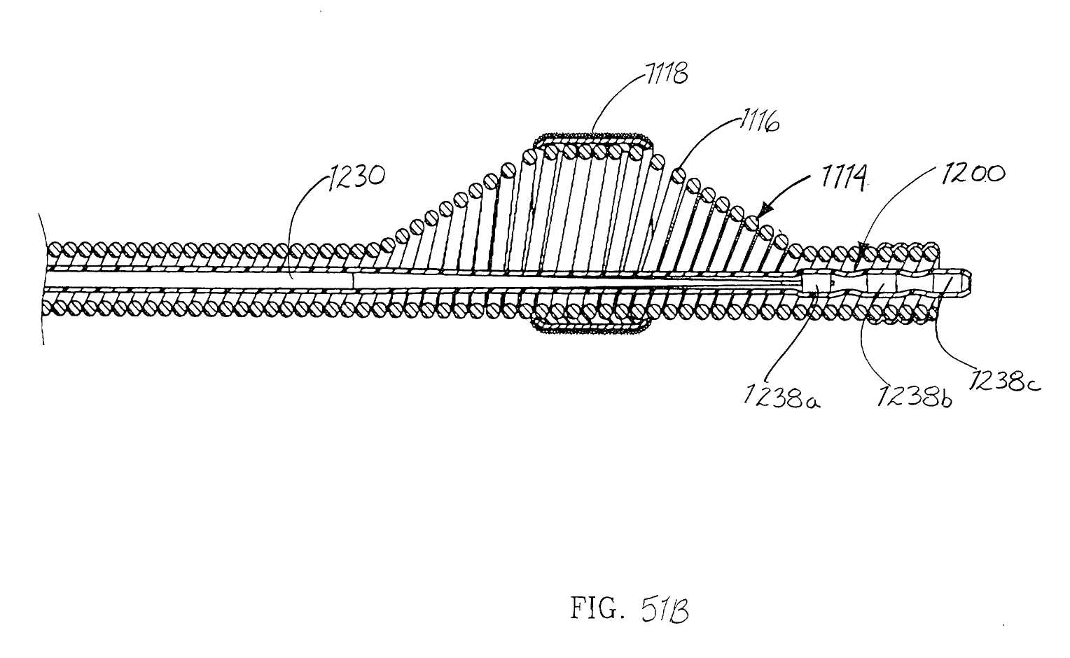

There is illustrated in FIG. 51, a prior art rotational atherectomy device 1100 as disclosed in a U.S. Pat. No. 6,132,444 to Shturman entitled “Eccentric Drive Shaft for Atherectomy Device and Method for Manufacture,” the disclosure of which is incorporated herein by reference in its entirety. In brief, atherectomy device 1100 includes an advancing device 1110 from which extends an elongated tubular sheath 1112. A flexible drive shaft 1114 formed from a helically wound coil extends through tubular sheath 1112 and has an enlarged eccentric coil section 1116 formed thereon at a location that is spaced from the distal end of the shaft. A ring 1118 extends about the outer periphery of the eccentric coil segment 1116 which is has a diamond coated abrasive surface deposited thereon.

As illustrated in FIG. 51, rotational atherectomy device 1100 is utilized in conjunction with a guidewire 1200 configured in accordance with a preferred embodiment of the subject invention. Referring to FIGS. 51A and 51B, guidewire 1200 includes a core wire 1230, a distal portion of which is conically tapered, preferably in a center-less grinding process. The tapering enhances the flexibility of the distal portion of core wire 1230 to reduce guidewire bias and a limit fatigue. The core wire 1230 is preferably formed from a metal alloy having shape memory characteristics. To enhance its flexibility, a distal portion of the core wire 1230 is heat-treated to relieve the shape memory characteristics associated therewith. As a result, the distal portion of the core wire 1230 is more compliant and less brittle than the untreated portion thereof. It is therefore more fatigue resistant than the untreated portion.

During an atherectomy procedure, a drive shaft is rotated about the axis of a guidewire at an extremely high rate of speed as the drive shaft is oscillated back and forth over the guidewire to advance through a lesion, as illustrated for example in FIGS. 51A and 51B. This causes extreme heat and friction, and can result in fatigue fractures in the guidewire. It is commonplace therefore, to introduce a saline cooling/lubricating solution between the relatively rotating components. An example of such a solution is one that is available from Boston Scientific and marketed under the tradename Rotaglide™ Lubricant.

The core wire 1230 of the subject invention is adapted and configured in such a manner so that a special lubricating solution such as Rotaglide™ Lubricant is wholly unnecessary. In particular, a distal portion of core wire 1230 is surrounded by a lubricious polymeric sleeve 1236. The sleeve creates has a low coefficient of friction between the outer surface of the core wire 1230 and the interior luminal surface of the drive shaft 1000. The polymeric sleeve 1236 is preferably formed from heat shrinkable polymer tubing, such as, for example, heat shrinkable polytetrafluoroethylene (PTFE) tubing.

A train of three spaced apart, generally cylindrical, radiopaque markers 1238a-1238c are positioned within sleeve polymeric 1236. The markers aide in the locational positioning of the distal end portion of guidewire 1200 within the vasculature of a patient when viewed by fluoroscopy or similar means. As illustrated, the outer diameter of each of the markers 1238a-1238c is greater than the minimal inner diameter of the interior lumen of sleeve 1236. Thus, the heat shrinkable sleeve 1236 secures the relative positions of the markers. The markers are preferably formed from a radiopaque material such as a platinum and iridium alloy.

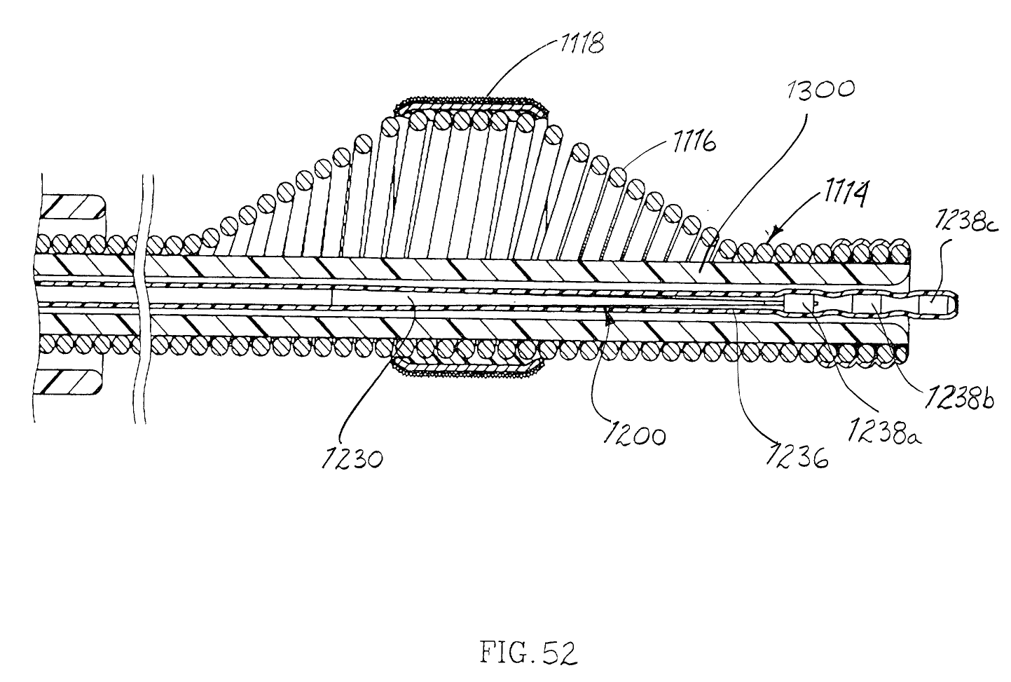

Referring now to FIG. 52, in accordance with a preferred embodiment of the subject invention, the drive shaft 1114 is provided with a flexible monolithic tubular liner 1300 formed from a lubricious material which reduces friction between the guidewire 1200 and the interior surface of drive shaft 1114, and thereby prevents the polymeric sleeve 1236 from abrading over time. The tubular liner 1300 can extend throughout the entire length of drive shaft 1114 or for a portion of its length sufficient to achieve the desired result. Preferably, the material from which tubular liner 1300 is formed is PTFE or High Density Polyethylene HDPE. The tubular liner 1300 is preferably glued to the interior surface of the drive shaft adjacent 1114 at the distal end thereof to secure its position. In the case of PTFE or HDPE, the outer surface of the tubular liner 1300 is preferably treated by plasma etching or a similar process to improve its ability to bond with the interior surface of the drive shaft. Alternatively, the tubular liner 1300 may be formed from a material such as PEBAX which bonds well to the coils of the drive shaft without the need for surface treatment.

The tubular liner 1300 has an outer diameter that is slightly larger than the inner diameter of the drive shaft 1114. Consequently, the outer diameter of the portion of the drive shaft containing the liner is increased. The inner diameter of tubular liner 1300 is greater than the outer diameter of the guidewire 1200 so that a gap exists between the two structures to allow free rotation of the drive shaft about the guidewire. Preferably, the interior lumen of liner 1300 is outwardly flared at its distal end to provide an atraumatic surface.

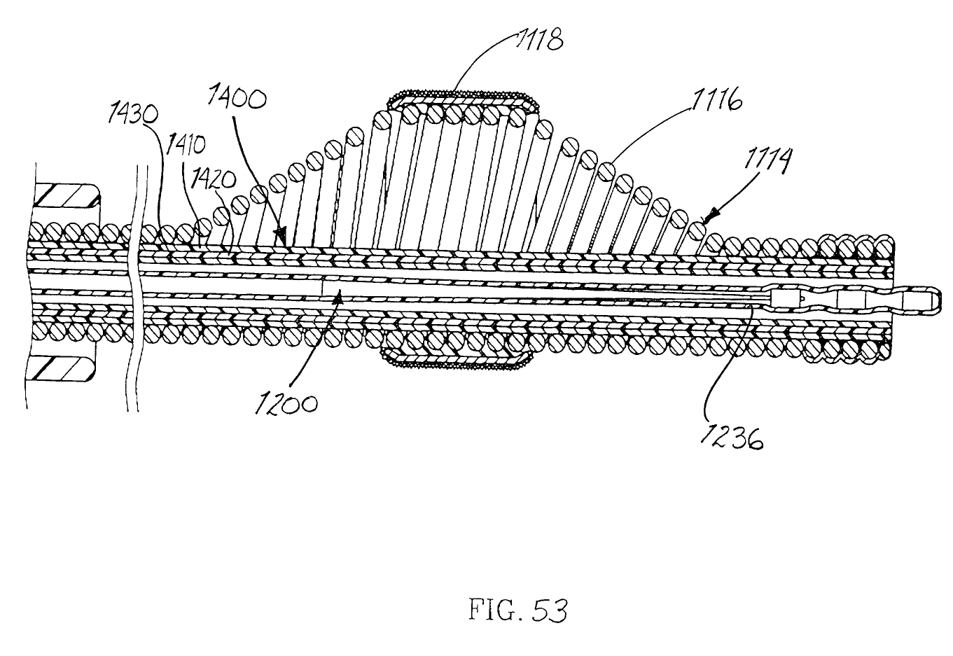

Referring to FIG. 53, in accordance with another preferred embodiment of the subject invention, drive shaft 1114 includes a three layer tubular liner 1400 formed in a coextrusion process. The three layers are preferably formed from dissimilar materials. More particularly, tubular liner 1400 includes an outer layer 1410 formed from a material that bonds well to the interior surface of the drive shaft and an inner layer 1430 formed from a lubricious material that reduces friction between the guidewire 1200 and the drive shaft 1114. The middle layer 1420 is a material that is selected to bond well with the inner and outer layers 1410 and 1430. Preferably, the outer layer 1410 of the liner is a material such as PEBAX and the inner layer 1430 of the liner is a material such as HDPE. A particularly suitable tubular liner 1400 is manufactured by Medical Extrusion Technologies, Inc. of Marrieta, Calif. This product has an outer diameter of about 0.0.019 in. and an inner diameter of about 0.015 in.

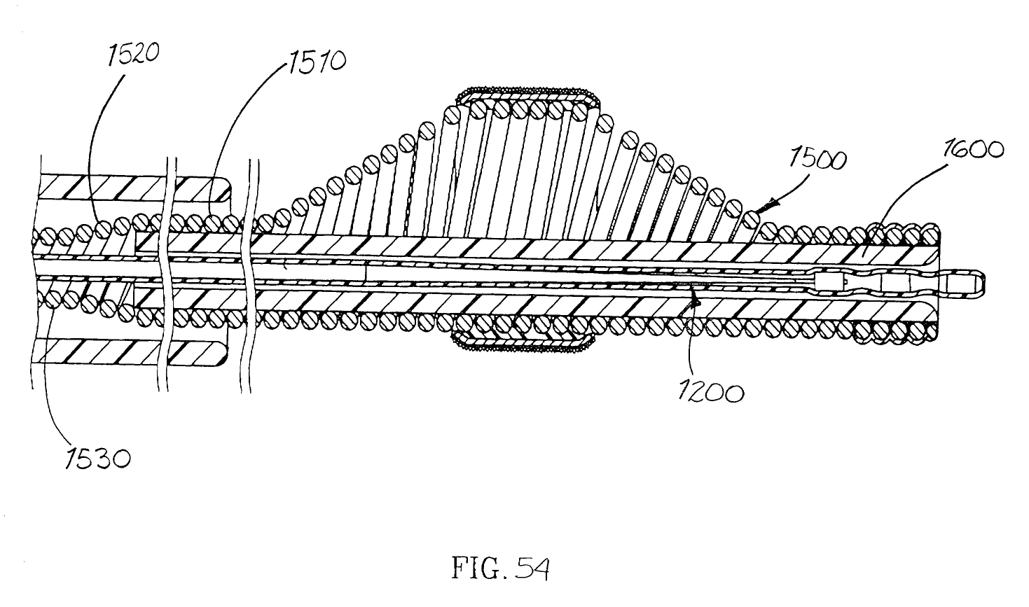

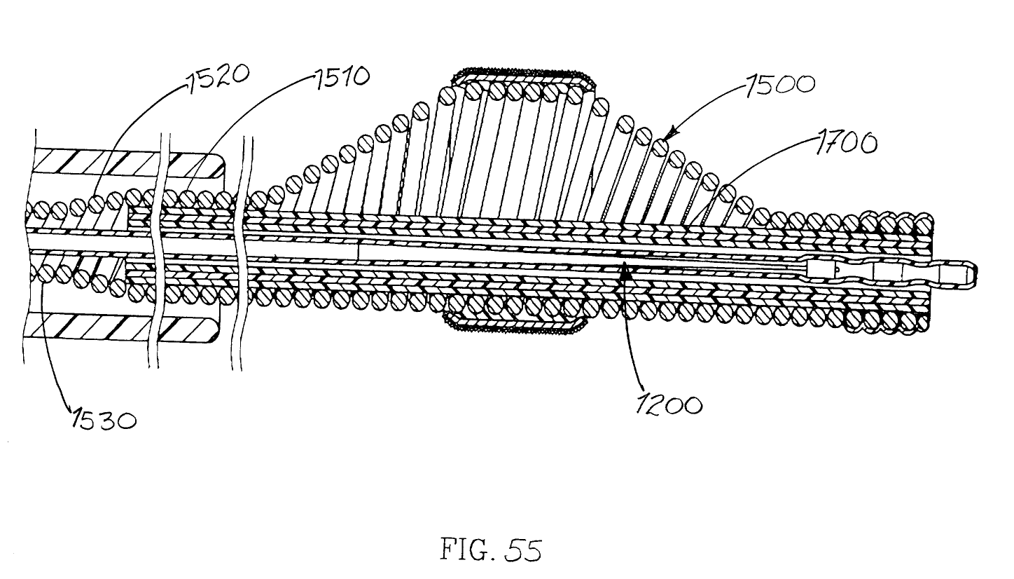

Referring now to FIGS. 54 and 55, there are illustrated additional embodiments of the drive shaft of the subject invention each designated generally by reference numeral 1500, and each having a conically tapered transitional segment 1520 delineating a distal section 1510 that contains a tubular liner and a proximal section 1530 that is without a tubular liner. Thus, the outer diameter of the distal section 1510 is greater than the outer diameter of the proximal section 1530. Preferably, the length of the distal section 1510 is about between 30 to 40 cm, which is sufficient to traverse the aortic arch and blood vessels distal thereto.

As illustrated in FIG. 54, drive shaft 1500 includes a monolithic tubular liner 1600 of predetermined length that is substantially similar to the monolithic tubular liner 1300. As illustrated in FIG. 55, drive shaft 1500 alternatively includes a three layer tubular liner 1700 of predetermined length that is substantially similar to the three layered tubular liner 1400. In each instance, the tubular liner 1600, 1700 are inserted into the distal section of the drive shaft 1500 so that it is properly seated therein. Thereafter, the distal end of the liner is bonded to the drive shaft to prevent its axial dislocation.

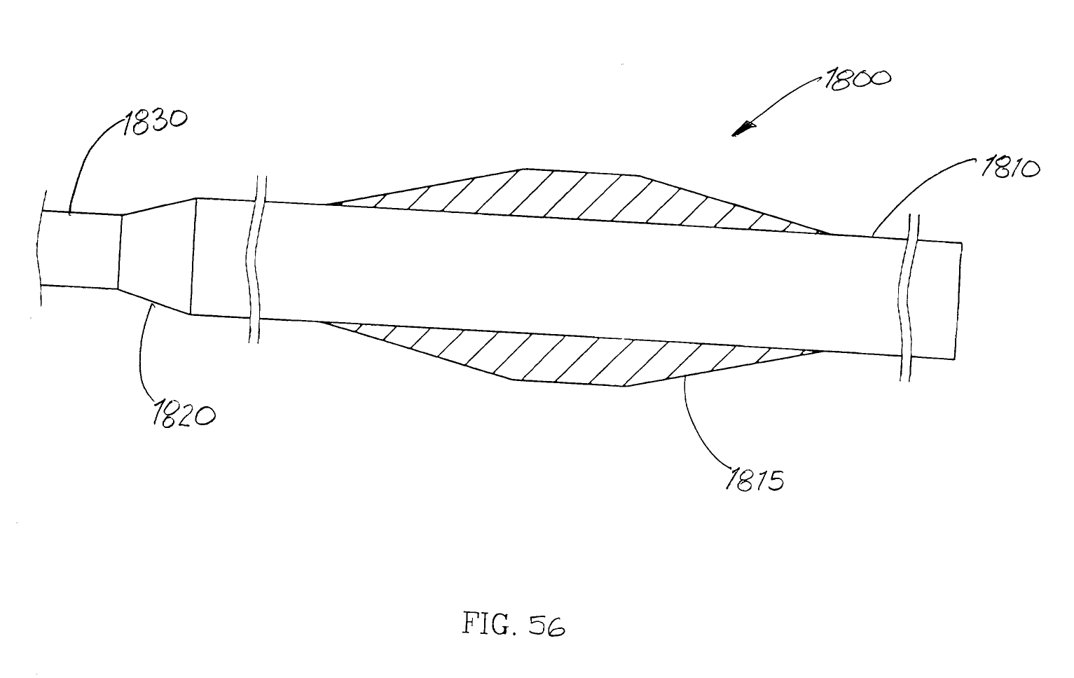

Referring to FIG. 56, there is illustrated a mandrel 1800 used to form the eccentric drive shaft 1500 of FIGS. 54 and 55. Mandrel 1800 has a distal section 1810 having an outer diameter of about 0.0175 in. about which the distal section 1510 of drive shaft 1500 is wound. Mandrel 1800 further includes a radially enlarged section 1815 about which the eccentric section of drive shaft 1500 is wound, and a conical section 1820 having a length of about 0.25 in. about which the tapered transition section 1520 of drive shaft 1500 is wound. Mandrel 1800 further includes a proximal section 1830 having an outer diameter of about 0.013 in. about which the proximal section 1530 of drive shaft 1500 is wound. The helically wound eccentric drive shaft 1500 is formed is a manner which is described in sufficiently enabling detail in U.S. Pat. No. 6,132,444 to Shturman, the disclosure of which has been previously incorporated by reference into the subject specification.

Although the high torque, low profile intravascular guidewire system of the subject invention and the methods disclosed herein have been described with respect to preferred embodiments, those skilled in the art will readily appreciate that changes and modifications may be made thereto without departing from the spirit and scope of the present invention.

What is claimed is:

1. An intravascular guidewire system comprising:
a) a core wire having an elongated wire body defining a relatively flexible distal portion and a relatively rigid medial portion; and
b) a torquing sheath for positioning the core wire, the torquing sheath having a normally curved, relatively flexible distal portion and an interior lumen dimensioned and configured to accommodate the core wire, wherein the medial portion of the core wire is sufficiently rigid to straighten the normally curved, relatively flexible distal portion of the torquing sheath when the medial portion of the core wire is extending therethrough.

2. An intravascular guidewire system as recited in claim 1, wherein the wire body is formed from a monofilament structure.

3. An intravascular guidewire system as recited in claim 1, wherein the wire body is formed from a metal alloy having shape memory characteristics.

4. An intravascular guidewire system as recited in claim 3, wherein the distal portion of the wire body is heat-treated in such a manner so as to relieve the shape memory characteristics associated therewith.

5. An intravascular guidewire system as recited in claim 1, wherein the distal portion of the wire body is conically tapered.

6. An intravascular guidewire system as recited in claim 5, wherein the distal portion of the wire body is conically tapered in a center-less grinding process.

7. An intravascular guidewire system as recited in claim 1, wherein the distal portion of the wire body is surrounded by a polymeric sheath.

8. An intravascular guidewire system as recited in claim 7, wherein the polymeric sheath extends beyond a distal end of the wire body to define a flexible tubular extension sleeve.

9. An intravascular guidewire system as recited in claim 7, wherein a clearance gap exist between the polymeric sheath and a distal-most section of the wire body.

10. An intravascular guidewire system as recited in claim 7, wherein the polymeric sheath is formed from heat shrinkable polytetrafluoroethylene tubing.

11. An intravascular guidewire system as recited in claim 7, wherein a plurality of spaced apart radiopaque markers are disposed within the tubular extension sleeve.

12. An intravascular guidewire system as recited in claim 11, wherein the radiopaque markers include at least three generally cylindrical markers formed from a platinum and iridium alloy.

13. An intravascular guidewire system as recited in claim 1, wherein the torquing sheath is constructed from plural helically wound layers of coiled wire filament.

14. An intravascular guidewire system as recited in claim 13, wherein the torquing sheath is constructed from an inner helically wound coil layer wound in a first direction and an outer helically wound coil layer wound in a second direction.

15. An intravascular guidewire system as recited in claim 1, wherein a tubular sleeve formed from a polymeric material is heat shrunk about the outer periphery of a proximal portion of the torquing sheath.

16. An intravascular guidewire system as recited in claim 15, wherein the tubular sleeve extends along about 15% to 40% of the length of the torquing sheath.

17. An intravascular guidewire system as recited in claim 1, wherein the curved distal portion of the torquing sheath has a reduced diameter to provide the curved distal portion with added flexibility and reduced rigidity relative to the remainder of the torquing sheath.

18. An intravascular guidewire system as recited in claim 17, wherein the reduction in diameter is accomplished by a chemical etching process.

19. An intravascular guidewire system as recited in claim 1, wherein the distal portion of the torquing sheath is electrically treated so as to set the curvature therefor.

20. An intravascular guidewire system as recited in claim 1, wherein the distal portion of the torquing sheath is defined by a flexible nosepiece having a radius of curvature.

21. An intravascular guidewire system as recited in claim 20, wherein the flexible nosepiece is formed from a polymeric material and includes a proximal portion for receiving the distal end of the torquing sheath.

22. An intravascular guidewire system as recited in claim 1, further comprising a rotatable ablation device including a drive shaft having an interior lumen and carrying an abrasive crown for ablating stenotic material, the interior lumen of the drive shaft being dimensioned and configured to receive the core wire.

23. An intravascular guidewire system as recited in claim 22, wherein a monolithic tubular liner formed from a lubricious material is disposed within the interior lumen of the drive shaft to reduce friction between the core wire and the drive shaft.

24. An intravascular guidewire system as recited in claim 22, wherein a three layer tubular liner is disposed within the interior lumen of the drive shaft, the liner having an outer layer formed from a material that bonds well to the interior surface of the drive shaft and an inner layer formed from a lubricious material that reduces friction between the guidewire and the drive shaft.

25. An intravascular guidewire system as recited in claim 22, wherein the drive shaft has an eccentric section carrying the abrasive crown and a conically tapered section proximal to an eccentric shaft section.

26. An intravascular guidewire comprising:
a core wire defined by an elongated wire body having opposed proximal and distal ends, a distal portion of the wire body being surrounded by a polymeric sheath, the polymeric sheath extending beyond the distal end of the wire body to define a flexible tubular extension sleeve.

27. An intravascular guidewire as recited in claim 26, wherein the wire body is formed from a monofilament structure.

28. An intravascular guidewire as recited in claim 26, wherein the wire body is formed from a metal alloy having shape memory characteristics.

29. An intravascular guidewire as recited in claim 28, wherein the distal portion of the wire body is heat-treated in such a manner so as to relieve the shape memory characteristics associated therewith.

30. An intravascular guidewire as recited in claim 26, wherein the distal portion of the wire body is conically tapered in a center-less grinding process.

31. An intravascular guidewire as recited in claim 26, wherein a clearance gap exist between the polymeric sheath and a distal-most section of the wire body.

32. An intravascular guidewire as recited in claim 26, wherein the polymeric sheath is formed from heat shrinkable polytetrafluoroethylene tubing.

33. An intravascular guidewire as recited in claim 26, wherein a plurality of spaced apart radiopaque markers are disposed within the tubular extension sleeve.

34. An intravascular guidewire as recited in claim 33, wherein the radiopaque markers include at least three generally cylindrical markers formed from a platinum and iridium alloy.

35. An intravascular guidewire as recited in claim 26, wherein a plug is provided at the distal end of the tubular extension sleeve.

36. An intravascular guidewire as recited in claim 26, wherein a coiled radiopaque marker wire is disposed within the tubular extension sleeve.

37. An intravascular guidewire comprising:
a core wire defined by an elongated wire body having opposed proximal and distal ends, the wire body formed from a metal alloy having shape memory characteristics, a distal portion of the wire body being heat treated in such a manner so to relieve the shape memory characteristics associated therewith.

38. An intravascular guidewire as recited in claim 37, wherein a polymeric sheath surrounds a distal portion of the wire body.

39. An intravascular guidewire as recited in claim 38, wherein the polymeric sheath extends beyond the distal end of the wire body to define a flexible tubular extension sleeve.

40. An intravascular guidewire as recited in claim 38, wherein the polymeric sheath is formed from heat shrinkable polytetrafluoroethylene tubing.

41. An intravascular guidewire as recited in claim 39, wherein a plurality of spaced apart radiopaque markers are disposed within the tubular extension sleeve.

42. An intravascular guidewire as recited in claim 41, wherein the radiopaque markers include at least three generally cylindrical markers formed from a platinum and iridium alloy.

43. An intravascular guidewire as recited in claim 39, wherein a coiled radiopaque marker wire is disposed within the tubular extension sleeve.

44. An intravascular guidewire as recited in claim 39, wherein a plug is provided at the distal end of the tubular extension sleeve.

45. An intravascular guidewire as recited in claim 39, wherein the distal portion of the wire body is conically tapered in a center-less grinding process.

46. An intravascular guidewire as recited in claim 45, wherein a clearance gap exist between the polymeric sheath and a distal-most section of the wire body.

47. An intravascular torquing sheath comprising:
an elongated body constructed from plural helically wound layers of coiled wire filament, including an inner helically wound coil layer wound in a first direction and an outer helically wound coil layer wound in a second direction, and having a curved distal portion.

48. An intravascular torquing sheath as recited in claim 47, wherein the curved distal portion of the torquing sheath has a reduced diameter to provide added flexibility and reduced rigidity.

49. An intravascular torquing sheath as recited in claim 48, wherein the reduction in diameter is accomplished by a chemical etching process.

50. An intravascular torquing sheath as recited in claim 47, wherein the distal portion of the torquing sheath is electrically treated so as to set the curvature therefor.

51. An intravascular torquing sheath as recited in claim 47, wherein the curved distal portion of the torquing sheath is defined by a flexible nosepiece having a radius of curvature.

52. An intravascular torquing sheath as recited in claim 51, wherein the flexible nosepiece is formed from a polymeric material and includes a proximal portion for receiving the distal end of the torquing sheath.

53. An intravascular torquing sheath as recited in claim 47, wherein a tubular sleeve formed from a polymeric material is heat shrunk about the outer periphery of a proximal portion of the torquing sheath.

54. An intravascular torquing sheath as recited in claim 53, wherein the tubular sleeve extends along about 15% to 40% of the length of the torquing sheath.

55. An intravascular torquing sheath as recited in claim 47, wherein a spherical member is secured to a distal end of the outer coil layer to render the distal end of the torquing sheath atraumatic to blood vessels.

56. An intravascular torquing sheath as recited in claim 55, wherein the spherical member is formed at least in part from a precious metal.

57. A method of forming an intravascular guidewire, comprising the steps of:
a) providing an elongated wire body formed from a material having shape memory characteristics; and
b) treating a distal portion of the wire body in a manner so as to relieve the shape memory characteristics associated therewith.

58. A method according to claim 57, wherein the step of treating a distal portion of the wire body includes heating a distal portion in an enclosure containing an inert gas for about thirty minutes at approximately 300° F.

59. A method according to claim 57, further comprising the step of heat shrinking a polymeric tube over a distal portion of the wire body.

60. A method of forming an intravascular torquing sheath, comprising the steps of:
a) providing an elongated body formed from plural helically wound coil wire layers;
b) positioning a distal portion of the elongated body over a cylindrical forming mandrel having a desired radius of curvature; and
c) delivering an electrical current through the distal portion of the elongated body such that the distal portion assumes the curvature of the forming mandrel.

61. A method according to claim 60, further comprising the steps of reducing the outer diameter of the distal portion of the torquing sheath to enhance the flexibility thereof.

62. A method according to claim 61, wherein the step of reducing the outer diameter of the distal portion of the torquing sheath includes masking the distal portion of the torquing sheath and applying a chemical etching agent thereto.
